(19) United States
(12) Patent Application Publication
(10) Pub. No.: US 2015/0196533 A1
(43) Pub. Date: Jul. 16, 2015

(54) METHOD FOR CONCURRENT TREATMENT OF PAIN AND DEPRESSION

(71) Applicant: The General Hospital Corporation, Boston, MA (US)

(72) Inventors: Jianren Mao, Lexington, MA (US); Hyangin Kim, Wellesley, MA (US)

(21) Appl. No.: 14/409,757

(22) PCT Filed: Jun. 28, 2013
(86) PCT No.: PCT/US2013/048418

§ 371 Date: Dec. 19, 2014

Related U.S. Application Data
(60) Provisional application No. 61/667,130, filed on Jul. 2, 2012.

(51) Int. Cl. A61K 31/405; A61K 45/06; A61K 31/138

(52) U.S. Cl. CPC A61K 31/405; A61K 31/138; A61K 45/06

(57) ABSTRACT

Provided herein are methods for the concurrent treatment of pain and depression in a patient based, at least in part, on the discovery that the comorbid interaction between pain and depression can be treated by regulating two opposing pathways of tryptophan metabolism.

### FEDERALLY SPONSORED RESEARCH OR DEVELOPMENT

This invention was made with Government support under Grant Nos. RO1DE18214, R01DE18538 and P20DA262002 awarded by the National Institutes of Health. The Government has certain rights in the invention.

### TECHNICAL FIELD

This invention relates to the concurrent treatment of both pain and depression, and more particularly to the concurrent treatment of both pain and depression through the modulation of brain indoleamine 2,3-dioxygenase (IDO1) activity.

### BACKGROUND

Epidemiological studies have consistently shown that chronic pain and depression are closely related in the clinical setting. The prevalence rate of depression is several times higher in patients with chronic pain than in a general population, whereas depression significantly increases the risk of developing chronic pain. Tricyclic antidepressants were initially used to treat pain related to trigeminal neuralgia, which raised the possibility that antidepressants may have a role in chronic pain treatment by increasing the content of monoamine neurotransmitters such as serotonin (5-HT) at synaptic sites. Currently, antidepressants and analgesics are often prescribed in combination for symptomatic management. This clinical approach has produced only limited success because it does not deal with the neural mechanism underlying the comorbid interaction between pain and depression.

While the comorbid relationship between pain and depression has long been recognized in the clinical setting, the cellular mechanism underlying the comorbid relationship remains unclear. Earlier studies have focused on a temporal relationship between pain and depression (e.g., antecedent hypothesis versus consequence hypothesis) but the cellular mechanism underlying this relationship remains unknown. Recent neurobiological studies have suggested that both depression and chronic pain may involve the monoaminergic system, the hypothalamic-pituitary-adrenal axis, as well as various other neurotransmitters/neuromodulators including acetylcholine, GABA, substance P, cholecystokinin, endogenous opioid, and brain-derived neurotrophic factor. Despite some progress, clinical treatment of pain and depression has so far been limited to symptomatic management. Thus, there remains a need to understand the regulatory mechanism underlying the comorbid interaction between pain and depression and to develop a new strategy for the concurrent treatment of both conditions.

Therefore, there is a need to develop a new treatment strategy that can concurrently alleviate pain and depression by regulating this interaction at the neuronal level. This approach is distinctively different from other approaches that involve the regulation of the immune system.

### SUMMARY

The present disclosure provides methods to target the cause of the comorbid interaction between pain and depression by regulating two opposing pathways of tryptophan metabolism. This new paradigm recognizes the necessity to dynamically balance two major tryptophan metabolic pathways in order to simultaneously alleviate both pain and depression, namely IDO1 and tryptophan hydroxylase (TPH) pathways.

Provided herein are methods for the concurrent treatment of pain and depression in a patient in need thereof, comprising administering to the patient an indoleamine 2,3-dioxygenase inhibitor. In one embodiment, the indoleamine 2,3-dioxygenase inhibitor is selected from 1-methyl-tryptophan (I-MT), β-(3-benzofuranyl)-alanine, β-(3-benzo(b)thienyl)-alanine, or 6-nitro-D-tryptophan.

Provided herein are methods of treating pain and depression in an patient comprising administering to the patient an effective amount of an indoleamine 2,3-dioxygenase inhibitor and a serotonin reuptake inhibitor to alleviate the pain and to alleviate at least one symptom of depression. In one embodiment, the indoleamine 2,3-dioxygenase inhibitor is selected from 1-methyl-tryptophan (I-MT), β-(3-benzofuranyl)-alanine, β-(3-benzo(b)thienyl)-alanine, or 6-nitro-D-tryptophan and the serotonin reuptake inhibitor is selected from citalopram, escitalopram, fluoxetine, sertraline, paroxetine, fluvoxamine, venlafaxine, duloxetine, dapoxetine, nefazodone, imipramin, femoxetine, indalpine, zimelidine, and clomipramine. The serotonin reuptake inhibitor may be a selective serotonin reuptake inhibitor (SSRI), a tricyclic antidepressant, or a mixed serotonin and norepinephrine reuptake inhibitor. In some embodiments, the pain is chronic pain selected from lower back pain, a typical chest pain, headache, pelvic pain, myofascial pain, abdominal pain, neck pain, neuropathic pain, fibromyalgia, and chronic pain caused by a disease or condition. In one embodiments, the methods further comprise administering tryptophan. In some embodiments, the symptom of depression is selected from agitation, restlessness, irritability, depressed mood, diminished interest and pleasure in activities, difficulty concentrating, weight and appetite changes, psychomotor disturbances, sleep disturbances, fatigue and loss of energy, feelings of hopelessness and helplessness, feelings of worthlessness and guilt, concentration difficulties, indecisiveness, thoughts of death or suicide and possibly delusions/hallucinations.

Provided herein are methods of treating a patient being diagnosed as having both pain and depression, the method comprising identifying a patient diagnosed with both pain and depression; administering to the patient an effective amount of an indoleamine 2,3-dioxygenase inhibitor and a serotonin reuptake inhibitor to alleviate the pain and to alleviate at least one symptom of depression. In one embodiment, the indoleamine 2,3-dioxygenase inhibitor is selected from 1-methyl-tryptophan (I-MT), β-(3-benzofuranyl)-alanine, β-(3-benzo(b)thienyl)-alanine, or 6-nitro-D-tryptophan and the serotonin reuptake inhibitor is selected from citalopram, escitalopram, fluoxetine, sertraline, paroxetine, fluvoxamine, venlafaxine, duloxetine, dapoxetine, nefazodone, imipramin, femoxetine, indalpine, zimelidine, and clomipramine. The serotonin reuptake inhibitor may be a selective serotonin reuptake inhibitor (SSRI). In some embodiments, the pain is chronic pain selected from lower back pain, a typical chest pain, headache, pelvic pain, myofascial pain, abdominal pain, neck pain, neuropathic pain, fibromyalgia, and chronic pain caused by a disease or condition. In some embodiments, the methods further comprise administering tryptophan. In some embodiments, the symptom of depression is selected from agitation, restlessness, irritability, depressed mood, diminished interest and pleasure in activities, difficulty concentrating, weight and appetite changes, psychomotor disturbances, sleep disturbances, fatigue and loss of energy, feelings of hopelessness and helplessness, feelings of worthlessness and guilt, concentration difficulties, indecisiveness, thoughts of death or suicide and possibly delusions/hallucinations.

Indoleamine 2,3-dioxygenase (IDO) is an enzyme that in humans is encoded by the IDO1 gene and functions as the first and rate-limiting enzyme in mammalian tryptophan metabolism. IDO catalyzes the oxidation of the essential amino acid tryptophan to N-formylkynurenine by dioxygen and is responsible for processing tryptophan in the human body.

Depression refers to an abnormal mood or a collection of symptoms that constitute a psychiatric disorder. Symptoms of depression include disturbances in mood and affect (agitation, restlessness, irritability, depressed mood, diminished interest and pleasure in activities, difficulty concentrating), bodily function (weight and appetite changes, psychomotor disturbances, sleep disturbances, fatigue and loss of energy), and cognitive processes (feelings of hopelessness and helplessness, feelings of worthlessness and guilt, concentration difficulties, indecisiveness, thoughts of death or suicide and possibly delusions/hallucinations). These symptoms vary in intensity, duration and frequency and permit classification of depression into different classes. Other symptoms of major depressive episodes include crying spells, self-pity, hopelessness, irritability, brooding, diminished self-esteem, decreased libido, nihilism, social withdrawal, memory impairment, feelings of inadequacy and pessimism. In some cases, depression can appear as anger and discouragement, rather than feelings of sadness.

Pain refers to a complex subjective sensation associated with actual or potential tissue damage and the affective response to it. Acute pain is a physiological signal indicating a potential or actual injury. Chronic pain may be divided into “nociceptive” (caused by activation of nociceptors), and “neuropathic” (caused by damage to or malfunction of the nervous system). Nociceptive pain is caused by stimulation of peripheral nerve fibers that respond only to stimuli approaching or exceeding harmful intensity (“nociceptors”). Neuropathic pain results from damage or disease affecting the nervous system.

Chronic pain includes lower back pain, a typical chest pain, headache, pelvic pain, myofascial pain, abdominal pain, or neck pain. Alternatively, chronic pain can be caused by a disease or condition such as arthritis, temporal mandibular joint dysfunction syndrome, traumatic spinal cord injury, disk herniation, multiple sclerosis, complex regional pain syndromes, irritable bowel syndrome, chronic fatigue syndrome, premenstrual syndrome, multiple chemical sensitivity, hyperventilation, closed head injury, fibromyalgia, rheumatoid arthritis, diabetes, cancer, HIV, or interstitial cystitis.

As used herein “treat” or “treating” refers to an amelioration of a disease or disorder, or at least one discernible symptom thereof. In another embodiment, “treatment” or “treating” refers to an amelioration of at least one measurable physical parameter, not necessarily discernible by the patient. In one embodiment, “treatment” or “treating” refers to inhibiting the progression of a disease or disorder, either physically, e.g., stabilization of a discernible symptom, physiologically, e.g., stabilization of a physical parameter, or both. In another embodiment, “treatment” or “treating” refers to delaying the onset of a disease or disorder.

As used herein “alleviate” or “alleviating” refers to lightening or lessening the severity of a symptom, condition, or disorder. For example, a treatment that reduces the severity of pain in a subject can be said to alleviate pain. It is understood that, in certain circumstances, a treatment can alleviate a symptom or condition without treating the underlying disorder. In certain aspects, this term can be synonymous with the language “palliative treatment.”

The term “diagnosed with” a condition refers to having been subjected to a physical examination by a person of skill, for example, a medical doctor (e.g., physician or veterinarian), and found to have the condition. It is also specifically contemplated that a subject (e.g., a mammal, a human) can be identified with such condition.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and FIGS., and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a set of six graphs showing the correlation of nociceptive and depressive behaviors. The injection of CFA (Complete Freund Adjuvant) into the right tibio-tarsal joint of Wistar rats produced mechanical allodynia (A) and thermal hyperalgesia (B) on the ipsilateral hindpaw as well as prolonged the immobility time in forced swimming test (C) and reduced the frequency (number of squares crossed) in open field test (D). Mean±S.E.M., n=10-11, *P<0.05, as compared with sham controls. FWL: foot-withdrawal latency. (E) The prolonged immobility time in forced swimming test was inversely correlated with the reduced hindpaw withdrawal latency in thermal hyperalgesia test. The data was obtained on day 14. Correlation coefficient: R=−0.913, n=10-11, *P<0.01. (F) The reduced frequency in open field test correlated with the reduced hindpaw withdrawal latency in thermal hyperalgesia test. Correlation coefficient: R=0.840, n=10-11, P<0.01.

FIG. 2 provides a set of photomicrographs and four bar graphs which show IDO1 expression in the hippocampus. (A) IDO1 immunoreactivity was detected in the hippocampus. Scale bar, 1.0 mm (left) and 50 μm (right). (B) Photomicrographs of colocalization between IDO1 and GFAP, Iba-1 or NeuN in the hippocampus. Scale bar: 50 μm. (C, D) IDO1 mRNA (C) and protein (D) expression was increased in the contralateral hippocampus of Wistar rats injected with CFA as detected by real-time PCR (C) and Western blot (D). day 0: baseline (naive rats); C-1 and C-14: samples taken on day 1 and day 14 from rats with CFA-induced arthritis; S-1 and S-14: samples taken on day 1 and day 14 from sham control rats. β-actin: loading control. Y-axis: fold change in IDO1 mRNA and protein expression. Mean±S.E.M., n=6-10, *P<0.05 as compared with sham control. (E, F) IDO1 expression (Western blot) was not up-regulated in the thalamus (E) or nucleus accumbens (F) in Wistar rats with CFA-induced arthritis, n=6 n P>0.05.

FIG. 3 is a set of bar graphs showing altered tryptophan metabolites by IDO enzyme activity. (A, B) The kynurenine (KYN)/tryptophan (TRP) ratio was increased (A), whereas the serotonin (5HT)/TRP ratio was decreased (B), in the contralateral hippocampus of Wistar rats with CFA-induced arthritis as assayed by HPLC. Mean±S.E.M., n=6, *P<0.05 as compared with sham control. (C, D) The KYN/TRP ratio (C) and the 5-HT/TRP ratio (D) were not changed in the thalamus of Wistar rats with CFA-induced arthritis as assayed by HPLC. The plasma KYN/TRP ratio (E), but not the 5-HT/TRP ratio (F), was increased in Wistar rats with CFA-induced arthritis, when both examined on day 14. Mean±S.E.M., n=6, *P<0.05 as compared with sham control. The plasma IDO level (G; ELISA) and the KYN/TRP ratio (H; HPLC), but not the 5-HT/TRP ratio (I; HPLC), was elevated in patients with both chronic back pain and depression. Mean±S.E.M., n=13-20, *P<0.05 as compared with healthy control.

FIG. 4 shows the relationship between anhedonic and nociceptive behavior. (A) The body weight gain was calculated as the percentage of the initial (baseline) body weight. Anhedonic rats gained less weight than control rats. (B) Sucrose preference was calculated as the percentage of the total fluid intake over 24 h. Sucrose preference was diminished in anhedonic rats. (C-F) The prolonged immobility time in FST (C; forced swimming test) and TST (D; tail suspension test) was associated with mechanical allodynia (E) and thermal hyperalgesia (F) in anhedonic rats. A-F: Mean+S.E.M, n=6 *P<0.05 as compared with control. (G-J) Mechanical allodynia (G) and thermal hyperalgesia (H) was exacerbated, along with a prolonged immobility time in FST (I) and TST (J) in anhedonic rats when examined at 7 days after the CFA-induced monoarthritis. (K) IDO1 protein expression was increased in anhedonic rats as compared with control rats with and without CFA injection. G-K: Mean+S.E.M, n=6 *P<0.05 as compared with sham control.

FIG. 5 is a set of graphs which show the effect of IDO 1 inhibition on behavioral changes. (A) Intraperitoneal injection of the IDO1 inhibitor L-1-MT (1-MT; 10 mg/d), given twice daily for 14 consecutive days beginning immediately after the CFA injection, attenuated mechanical allodynia on the ipsilateral hindpaw of rats. (B) The same 1-MT treatment regimen concurrently improved the immobility time in forced swimming test in the same rats. Mean±S.E.M., n=6, *P<0.05 as compared with vehicle control. (C, D) Intra-hippocampal microinjection of 1-MT (5 □g in 0.5 □l), once daily for 7 days beginning immediately after the CFA injection, also attenuated thermal hyperalgesia (C) on the ipsilateral hindpaw of rats as well as reduced the immobility time in forced swimming test (D) in the same rats. Mean±S.E.M., n=6, *P<0.05 as compared with vehicle control. FWL: foot-withdrawal latency. (E) IDO1 expression in the hippocampus was decreased in rats receiving a 14-day intraperitoneal administration of 1-MT (10 mg/d). day 0: baseline (naive rats): C-1 and C-14: samples taken on day 1 and day 14 from rats with CFA-induced arthritis; S-1 and S-14: samples taken on day 1 and day 14 from control rats. β-actin: loading control. Mean±S.E.M., n=6, *P<0.05 as compared with sham control. The same systemic 1-MT treatment regimen reduced the kynurenine (KYN)/tryptophan (TRP) ratio (F), and increased the serotonin (5-HT)/TRP ratio (G), in the hippocampus. Mean±S.E.M., n=6, *P<0.05 as compared with vehicle control.

FIG. 6 is a set of graphs which show the effect of IDO knockout on behavioral changes. (A) IDO knockout mice had no IDO1 mRNA expression (real-time PCR) in the hippocampus. The IDO1 mRNA expression in wild-type mice was increased in the hippocampus after the CFA injection. Mean±S.E.M., n=6, *P<0.05 as compared with sham control. (B. C) Mechanical allodynia (B) and thermal hyperalgesia (C) on the ipsilateral hindpaw were attenuated in IDO knockout mice. (D, E) IDO knockout also reduced the immobility time in forced swimming test (FST) (D) and the decreased frequency in open field test (E) in the same mice with CFA-induced arthritis. FWL: foot-withdrawal latency. Mean±S.E.M., n=6, *P<0.05 as compared with wild-type mice. (F-G) Intraperitoneal injection of acetaminophen (APAP; 100 mg/kg), given once on day 14, attenuated ipsilateral mechanical allodynia (F) and thermal hyperalgesia (G) when examined at 1 hr after the injection. Mean±S.E.M., n=6, *P<0.05 as compared with vehicle control. (H) The same APAP treatment did not change the immobility time in FST in the same rats. (I) The contralateral hippocampal IDO1 mRNA expression was increased after the CFA injection, which was not reversed by a single APAP treatment. H, I: Mean±S.E.M., n=6, P>0.05, as compared with vehicle control.

FIG. 7 is a set of graphs which show the role of IL-6 in the hippocampal JAK-STAT pathway. (A, B) The plasma IL-6 concentration was increased (A, ELISA), so was the IL-6 mRNA expression in the hippocampus (B, real-time PCR), in rats with CFA-induced arthritis. (C) The IL-6 mRNA expression was also increased in the hippocampus of both IDO knockout and wild-type mice with CFA-induced arthritis. Mean±S.E.M., n=6, *P<0.05 as compared with sham control. (D) The plasma IL-6 level was elevated in patients with both chronic back pain and depression (ELISA). Mean±S.E.M., n=13-20, *P<0.05 as compared with healthy control. (E-G) The expression of JAK2 (E), STAT3 (F), and p-STAT3 (G) in the hippocampus was increased in rats with CFA-induced arthritis (Western blot). C-1, C-7 and C-14: samples taken on day 1, 7 and 14 from rats with CFA-induced arthritis, S-1, S-7 and S-14: samples taken on day 1, 7 and 14 from sham control rats. β-actin: loading control. Mean±S.E.M.; n=4-5, *P<0.05 as compared with sham control.

FIG. 8 provides a set of photomicrographs and four bar graphs which show the effects of IL-6 on IDO1 expression in Neuro2a cells and organotypic hippocampal culture. (A) IDO1 immunoreactivity was increased in IL-6-treated Neuo2a cells, as expressed in perinuclear cytoplasm when co-stained with DAPI. Scale bar. 50 □m. (B-E) The IDO1 mRNA (B) and protein (C) expression, as well as IDO activity [kynurenine(KYN)/tryptophan(TRP) ratio (D) and serotonin(5-HT)/TRP ratio (E)], was increased in cultured Neuro2a cells after adding IL-6 (0.5 ng/ml) for 24 hours. (F-H) Exposure of exogenous IL-6 (100 ng/ml) for 24 hours increased the expression of IDO1 (F: immunoreactivity; G: mRNA; H: Western blot) in hippocampal organotypic slice culture. Scale bar 500 um (upper panel) and 50 um (lower panel). In addition, adding IL-6 for 24 hours also increased KYN/TRP ratio and decreased 5-HT/TRP ratio (HPLC) in the culture medium (I). * P<0.05, as compared with vehicle control.

FIG. 9 is a set of graphs which show the role of IL-6 signaling in hippocampal IDO expression and behavioral changes. (A-D) Microinjection of an IL-6 antiserum (IL-6 Ab; 0.5 μg/day; once daily×7d) into the hippocampus contralateral to the hindpaw with CFA-induced arthritis attenuated mechanical allodynia (A) and thermal hyperalgesia (B) on the ipsilateral hindpaw without change in nociceptive threshold in sham rats (data not shown). The same IL-6 antiserum microinjection regimen concurrently improved depressive behavior in forced swimming test (C) and prevented the hippocampal IDO1 up-regulation (D) in these same rats. day 0: baseline (naive rats); Ab-1 and Ab-7: samples taken on day 1 and day 7 from Wistar rats treated with IL-6 antiserum. V-1 and V-7: samples taken on day 1 and day 7 from Wistar rats treated with control serum. β-actin: loading control. Mean±S.E.M., n=6, *P<0.05 as compared with vehicle (control serum). FWL: foot-withdrawal latency. (E-H) Microinjection of exogenous IL-6 (0.1 μg/0.5 μl; once daily×7d) into the left hippocampus of naive rats (without CFA injection) induced mechanical allodynia (E) and thermal hyperalgesia (F) on the right hindpaw. The same IL-6 microinjection regimen concurrently induced depressive behavior in forced swimming test (G) and up-regulated the hippocampal IDO1 mRNA expression (H) in these same rats. The effects from the intra-hippocampal microinjection of IL-6 were blocked when IL-6 was co-administered with AG490 (a JAK/STAT inhibitor, 5 μg/0.5 μl) for 7 days. Mean±S.E.M., n=4-5, *P<0.05 as compared with vehicle control.

FIG. 10 is a set of graphs which show the effect of drug treatment on nociceptive and depressive behaviors as well as the regulation of IDO1/TPH2 ratio in an animal pain model of combined pain and depression. Rats were divided into five groups: 1) sham-operated rats treated with a vehicle (Sham-V), 2) CFA-induced hindpaw pain treated with vehicle (CFA-V), 3) CFA-induced hindpaw pain treated with fluoxetine (5 mg; CFA-SSRI or SSRI), 4) CFA-induced hindpaw pain treated with the IDO inhibitor 1-MT (10 mg, CFA-1MT or 1MT), and 5) CFA-induced hindpaw pain treated with both fluoxetine (5 mg) and 1-MT (10 mg) (CFA-SSRI+1MT or SSR+1MT). Behavioral tests for pain are shown in panel A (mechanical allodynia) and panel B (thermal hyperalgesia). Behavioral tests for depression are shown in panel C (forced swimming test) and panel D (tail-suspension test). Day (−3) and Day (−7) indicate the time point at 3 and 7 days after discontinuation of the corresponding drug treatment.

FIG. 11 provides a set of photomicrographs and four bar graphs which show IDO1/TPH2 expression in the hippocampus. Rats were divided into five groups: 1) sham-operated rats treated with a vehicle (Sham), 2) CFA-induced hindpaw pain treated with vehicle (C-V), 3) CFA-induced hindpaw pain treated with fluoxetine (5 mg; C-S or C-SSRI), 4) CFA-induced hindpaw pain treated with the IDO1 inhibitor 1-MT (10 mg, C-1MT or C-MT), and 5) CFA-induced hindpaw pain treated with both fluoxetine (5 mg) and 1-MT (10 mg) (C-S+MT or C-S+1MT). Each treatment was given intraperitoneally once per day from day 1 to day 14.

### DETAILED DESCRIPTION

The methods described herein are based, at least in part, on the discovery that the comorbid interaction between pain and depression can be treated by manipulating two opposing pathways of tryptophan metabolism. More specifically, the methods described herein, are based, at least in part, on the recognition that by dynamically balancing two major tryptophan metabolic pathways, namely IDO1 and tryptophan hydroxylase (TPH) pathways, it is possible to simultaneously alleviate both pain and depression. Balancing the content of endogenous neurotransmitters through the regulation of tryptophan metabolism, which enhances therapeutic effectiveness, may reduce side effects and improve patient compliance.

Tryptophan is an essential amino acid and the precursor of serotonin and kynurenine, two neuromodulators critically implicated in the regulation of neuronal excitation and depression. Indoleamine 2,3-dioxygenase (IDO) is a rate-limiting enzyme in tryptophan metabolism. Kynurenine and serotonin are two major tryptophan metabolites via enzymatic regulation including IDO. Relative to its basal expression in immune cells, IDO is significantly up-regulated in response to inflammation. Recent studies in the immunology fields have shown that IDO activity is linked to 1) a decreased serotonin content and depression and 2) an increased kynurenine content and neuroplastic changes through the effect of its derivatives such as quinolinic acid on glutamate receptors. Moreover, IDO expression has been shown to be induced by proinflammatory cytokines leading to the increased kynurenine production.

Serotonin (5-HT) is a product of tryptophan metabolic pathway, which is directly regulated by TPH2 [(a neuronal form of tryptophan hydroxylase (“TPH”)] and indirectly regulated by IDO1 (a neuronal form of IDO). When the expression of IDO1 is increased without a concurrent change in the expression of TPH, as demonstrated under a chronic pain condition, tryptophan metabolism will be shifted away from the production of 5-HT. As such, the IDO1/TPH2 ratio is an important regulatory target in order to keep the tryptophan metabolism towards the production of 5-HT. This can be achieved by selectively inhibiting IDO1 activity and/or increasing TPH2 expression according to the methods described herein.

Although using antidepressant alone can increase the 5-HT content in the synaptic cleft, the pre-synaptic 5-HT production and storage will be depleted if IDO1 is up-regulated in the setting of comorbid chronic pain without a concurrent increase in TPH2 expression. This leads to a clinical phenomenon of antidepressant tachyphylaxis (lack of effect following repeated use) with its diminished effect on pain and depression. Selectively inhibiting IDO1 alone or in combination with an antidepressant, preferably a selective serotonin reuptake inhibitor (SSRI), allows for regulating the balance between the pre-synaptic 5-HT production and storage and post-synaptic 5-HT availability, thereby preventing antidepressant tachyphylaxis and enhancing the relief of both pain and depression Kynurenine is a major metabolic product of IDO1, which has a negative regulatory effect on pain and depression. Up-regulation of IDO1 directly increases the kynurenine content and indirectly decreases the serotonin content through tryptophan depletion, thereby raising the kynurenine over serotonin ratio. Up-regulation of IDO1, when accompanied by down-regulation or no change of TPH2 expression, will further shift the balance towards the increased kynurenine over serotonin ratio. This again can be regulated by using an IDO1 inhibitor, alone or in combination with other agents.

Melatonin and quinolinic acid are metabolic products of serotonin and kynurenine, respectively. While melatonin helps improve chronic pain, quinolinic acid contributes to neuronal plasticity and chronic pain. Therefore, up-regulation of IDO1 under a chronic pain condition can result in the decreased melatonin but increased quinolinic acid level, further contributing to the comorbid interaction between pain and depression.

### IDO1 Inhibitors

Skilled practitioners will appreciate that many art-known commercially available IDO inhibitors are suitable for use in the methods disclosed herein. Non-limiting examples of IDO inhibitors include 1-methyl-tryptophan (I-MT), β-(3-benzofuranyl)-alanine, β-(3-benzo(b)thienyl)-alanine, 6-nitro-D-tryptophan, and combinations thereof.

### Serotonin Reuptake Inhibitors

Serotonin reuptake inhibitors (SRI) are a type of drug that acts as a reuptake inhibitor for the neurotransmitter serotonin (5-hydroxytryptamine (5-HT)) by blocking the action of the serotonin transporter (SERT). SRIs are used predominantly as antidepressants, though they are also commonly used in the treatment of other psychological conditions such as anxiety disorders and eating disorders. Skilled practitioners will appreciate that SRIs can either be selective or non-selective depending on their action. Selective serotonin reuptake inhibitors or serotonin-specific reuptake inhibitor (SSRIs) are a class of compounds typically used as antidepressants in the treatment of depression, anxiety disorders, and some personality disorders. Non-limiting examples of serotonin reuptake inhibitors include citalopram, escitalopram, fluoxetine, sertraline, paroxetine, fluvoxamine, venlafaxine, duloxetine, dapoxetine, nefazodone, imipramin, femoxetine, indalpine, zimelidine, and clomipramine. Additional examples of serotonin reuptake inhibitors are known in the art. Reference to a particular serotonin reuptake inhibitor such as “citalopram” is intended to include any pharmaceutically acceptable form, such as salts of that compound. Further, each of the serotonin reuptake inhibitors specified above is intended to be an individual embodiment. Accordingly, each of them and the use thereof may be used individually. Alternatively, the serotonin reuptake inhibitor may also be a combination of two or more different serotonin reuptake inhibitors.

In a further aspect, the serotonin reuptake inhibitor is a selective serotonin reuptake inhibitor or serotonin-specific reuptake inhibitor (SSRIs).

### Methods of Treatment

The methods described herein include methods for the treatment (e.g., concurrent treatment) of pain and symptoms associated with depression. In some embodiments, the pain is chronic pain and the symptoms associated with depression include, but are not limited to, agitation, restlessness, irritability, depressed mood, diminished interest and pleasure in activities, difficulty concentrating, weight and appetite changes, psychomotor disturbances, sleep disturbances, fatigue and loss of energy, feelings of hopelessness and helplessness, feelings of worthlessness and guilt, concentration difficulties, indecisiveness, thoughts of death or suicide and possibly delusions/hallucinations

Generally, the methods include administering a therapeutically effective amount of an indoleamine 2,3-dioxygenase inhibitor as described herein, to a subject who is in need of, or who has been determined to be in need of, such treatment. Additionally, the methods include administering a combination of a therapeutically effective amount of an indoleamine 2,3-dioxygenase inhibitor as described herein and a therapeutically effective amount of a serotonin reuptake inhibitor. The methods contemplated herein also include administering a therapeutically effective amount of an indoleamine 2,3-dioxygenase inhibitor as described herein and tryptophan; administering a therapeutically effective amount of a serotonin reuptake inhibitor with an antagonist of one or more major IDO products; administering a combination of a serotonin reuptake inhibitor with an inhibitor of an enzyme or intracellular element involved in the metabolism of an IDO product; or administering a combination of an indoleamine 2,3-dioxygenase inhibitor as described herein with a serotonin receptor agonist.

Monitoring is an important component to effective treatment of pain and depression. Clinical assessments and patient interviews are commonly used for diagnosing and monitoring treatment of patients with pain and depression. Once a patient has been treated for pain and depression according to the methods provided herein, the patient is then monitored for depression symptoms by conventional analysis.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions and Methods of Administration

The methods described herein include the manufacture and use of pharmaceutical compositions, which include compounds identified by a method described herein as active ingredients. Also included are the pharmaceutical compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin, an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

### EXAMPLES

The invention is further described in the following examples, which do not limit the scope of the invention described in the claims.

### Experimental Animals

Male Wistar rats (250-300 gm; Charles River) or 7-10 week old male B6.129-Ido1^{tmlAlm}/J(IDO^{-/-}) and C57BL/6J wild-type (WT) mice (Jackson Laboratory) were used. Animals were housed individually (21±2° C., relative humidity 50±10%, 12 h light/dark cycle) with water and food available ad libitum.

### Surgical Procedures

Hindpaw Monoarthritis:

Hindpaw monoarthritis was induced by the injection of CFA (50 μl) into a unilateral tibio-tarsal joint cavity under brief isoflurane anesthesis (Butler, S. H., et al, and Weil-Fugazza, J. 1992. A limited arthritic model for chronic pain studies in the rat. Pain 48:73-81). Animals in sham groups received the injection of 50 μl Incomplete Freund's Adjuvant. Local redness and joint swelling were observed in CFA-injected rats but not control rats during the experimental period.

Brain Cannula Implantation and Drug Injection:

Under brief isoflurane anesthesia, a guide cannula (26 gauge, Plasticsone) was implanted just above the hippocampus (AP: −3.6 mm; left: +2.0 mm from Bregma; depth: −4.0 mm from skin) (Paxinos, G., and Watson, C. 1998. The rat brain in stertaxic coordinates. New York: Academic Press). An injection needle (33 gauge, Plasticsone) was inserted through the guide cannula and drug solution or vehicle (0.5 μl) was slowly injected over 5 min using a Hamilton syringe. Locations of the cannula placement were confirmed at the time of tissue harvest (data not shown).

### Anhedonia Induced by Chronic Social Stress

To induce anhedonia-like behavior in rats, chronic social stress was introduced using a modified resident-intruder social interaction method as described previously (Krsiak, M. 1975. Timid singly-housed mice: their value in prediction of psychotropic activity of drugs. Br. J. Pharmacol. 55:141-150; Strekalova, T., et aL, 2004. Stress-induced anhedonia in mice is associated with deficits in forced swimming and exploration. Neuropsychopharmacology 29:2007-2017; Rygula, R., et al., 2005. Anhedonia and motivational deficits in rats: impact of chronic social stress. Behav. Brain Res. 162:127-134) An experimental rat (275-300 gm), named as an intruder rat in this model, was transferred from its home cage into a cage of a resident rat (500-600 gm) for one hour per day. The intruder rat and the resident rat were separated by a small round wire-mesh compartment (diameter 11 cm, height 14 cm) within the resident cage. After one hour, the intruder rat was returned to its home cage. This procedure was carried out at the beginning of the dark cycle in a 24-hour light cycle (light on and off for each 12 hour-period). An intruder rat was confronted with a different resident rat each day. This process lasted for four weeks. For controls, rats were placed in the same behavioral room but without the social interaction with a resident rat.

### Behavioral Tests

Nociceptive Test:

Animals were habituated to a test setting (30-min session) for three consecutive days. Thermal withdrawal threshold was assessed using the Hargreaves' apparatus and method (Hargreaves, K., et al., 1988. A new and sensitive method for measuring thermal nociception in cutaneous hyperalgesia. Pain 32:77-88). Mechanical withdrawal threshold was assessed using von Frey filaments (Tal, M., and Bennett, G. J. 1994. Extra-territorial pain in rats with a peripheral mononeuropathy: mechano-hyperalgesia and mechano-allodynia in the territory of an uninjured nerve. Pain 57:375-382).

Forced Swim Test (FST):

FST was performed according to the original method by Porsolt et al (Porsolt, R. D., Le, P. M., and Jalfre, M. 1977. Depression: a new animal model sensitive to antidepressant treatments. Nature 266:730-732). The total duration of immobility (non-swimming) within a 5-min session was recorded as immobility scores (in seconds) and compared among groups.

Tail Suspension Test (TST):

Tail suspension test was carried out as described previously (Steru, L., Chermat, R., Thierry, B., and Simon, P. 1985. The tail suspension test: a new method for screening antidepressants in mice. Psychopharmacology (Berl) 85:367-370). Briefly, a rat was hung on a hook, using adhesive tape placed 20 mm from the tip of its tail, on a 50 cm-height rod. The immobility time during a 6-min period of tail suspension was recorded with a stopwatch.

Open Field Test (OFT):

OFT was carried out in a plexiglas square box (57 cm×57 cm) with walls of 50 cm height (Denenberg, V. H. 1969. Open-field behavior in the rat: what does it mean? Ann. N. Y. Acad. Sci. 159:852-859). Behaviors were observed for 10 min under a dim light. After the first 5 min (habituation), the number of squares crossed by a rat was recorded for the next 5 minutes.

Sucrose Preference Test (SPT):

Sucrose preference test was performed as described in previous studies (Willner, P., et al., 1987. Reduction of sucrose preference by chronic unpredictable mild stress, and its restoration by a tricyclic antidepressant. Psychopharmacology (Berl) 93:358-364; Sigwalt, A. R., et al., 2011. Molecular aspects involved in swimming exercise training reducing anhedonia in a rat model of depression. Neuroscience 192:661-674). To perform this test, 1% sucrose solution was offered once weekly in a rat's home cage. Each rat had free choices between 1% sucrose solution bottle and a tap water bottle. Sucrose and tap water intakes were separately measured by weighing each bottle before and after the test (a 24-hour period). The sum of tap water and sucrose water intake (in grams) was calculated as the total water intake. The sucrose preference was expressed as the percentage of sucrose water intake from the total water intake.

Rotarod Test:

A rotarod test, using an accelerating rotarod apparatus (Columbus Instruments), was performed in both arthritic and sham control rats. Rats were given three training sessions and then placed on a 9-cm-diameter rod with the increased speed from 0.5 rpm to 30 rpm over a 60-s period. Each rat was tested in three consecutive trials with 15 min intervals. The duration of time on the rotarod was determined automatically by a timer that recoded to the nearest second.

### Cultures

Neuro2a Cell Culture:

Neuro2a cells were cultured in minimal essential (EAGLE) medium (Invitrogen) supplemented with 10% fetal bovine serum, 100 U/ml penicillin, and 100 μg/ml streptomycin (Invitrogen) in a humidified atmosphere of 5% CO₂ and 95% air at 37° C. Cells were treated with 0.5 ng/ml IL-6 or vehicle and collected 24 hours later for assays (74).

Organotypic Hippocampal Tissue Culture:

According to a modified method (Stoppini, L., Buchs, P. A., and Muller, D. 1991. A simple method for organotypic cultures of nervous tissue. J Neurosci. Methods 37:173-182.; Fu, X., Zunich, S. M., O'Connor, J. C., Kavelaars, A., Dantzer, R., and Kelley, K. W. 2010. Central administration of lipopolysaccharide induces depressive-like behavior in vivo and activates brain indoleamine 2,3 dioxygenase in murine organotypic hippocampal slice cultures. J Neuroinflammation. 7:43) organotypic hippocampal cultures were prepared using hippocampus slices from 5 to 8-day-old Wistar rats. Rat pups were euthanized by decapitation and brains were rapidly removed and the hippocampus was separated from the brain. Hippocampal slices (350 μm in thickness) were made using a Mcllwain tissue chopper (Campden Instruments Ltd) and placed in the Gey's balanced salt solution (Sigma-Aldrich) with 2 mg/ml D-glucose. Slices were then placed on 30-mm Millicell-CM (Millipore) porous (0.4 μm) membranes. These membranes were transferred into six-well culture plates filled with an incubation medium (1 ml) consisting of 50% vol/vol minimal essential medium, 25% heat-inactivated horse serum and 25% HBSS (Invitrogen), supplemented with 25 mM D-glucose. Plates were incubated in a humidified atmosphere of 5% CO₂ and 95% air at 37° C. Culture medium was changed 3 times a week. Slices were treated with 100 ng/ml IL-6 or vehicle and collected 24 hours later for assays (Hakkoum, D., Stoppini, L., and Muller, D. 2007. Interleukin-6 promotes sprouting and functional recovery in lesioned organotypic hippocampal slice cultures. J Neurochem. 100:747-757).

### Immunohistochemistry

Rats were anesthetized with pentobarbital (50 mg/kg, i.p.) and perfused transcardially with cold 0.9% saline (pH 7.4±0.1) followed by cold 4% paraformaldehyde in a phosphate buffer (PB, 0.1M, pH 7.4±0.1). Brains were removed and placed in the same fixative for post-fixation at 4° C. for 6 hours. For cryoprotection, the samples were kept with cold 30% sucrose in phosphate buffer saline (PBS) at 4° C. until samples sank to the bottom. Coronal brain sections (30 μm) were cut using a cryostat and then floated in PBS. Sections were blocked with 5% normal donkey serum in 0.3% Triton X-100/PBS for 1 hour at room temperature. Free floating sections were incubated overnight at 4° C. on a rocker with one of the following primary antibodies: 1:100, IDO1 rabbit polyclonal (Santa Cruz Biotechnology Inc.); 1:1,000, NeuN mouse monoclonal (Chemicon); 1:1,000, GFAP mouse monoclonal (BD Bioscience Pharmingen); 1:1,000, Iba-1 mouse monoclonal (Abcam). Sections were then washed with PBS three times and incubated with 1:300 FITC- or cyanine3-conjugated secondary antibody (JacksonlnununoResearch Laboratories Inc) for 1 hour at room temperature. Blue fluorescent DAPI (1:300; Invitrogen) was used to stain the nucleus of fixed cultured Neuro2a cells for 15 minutes.

Colocalization was examined by adding a second primary antibody following the same procedure as described above. Controls were performed by either omitting primary antibody or using antigen absorption (data not shown). Images were examined with a fluorescence microscope (Olympus) and captured with a digital camera. The images were analyzed using Adobe PhotoShop (Version 7).

### Western Blot

Rats were sacrificed under pentobarbital anesthesia. The hippocampus was separated from the brain and immediately stored on dry ice and kept at −80° C. until use. Samples were homogenized in a lysis buffer with SDS containing a cocktail of proteinase inhibitors (Roche). Samples were separated on an SDS-PAGE gel (4%-15% gradient gel; Bio-Rad) and transferred to polyvinylidene difluoride filter (PVDF) membranes (Millipore). Membranes were blocked with 5% nonfat dried milk for 1 hour at room temperature and incubated over night at 4° C. on a rocker with one of following primary antibody: 1:200 IDO1 (Santa Cruz Biotechnology Inc.; rabbit polyclonal antibody), 1:200 IDO1 (Novius Biologicals; rabbit polyclonal antibody), 1:500 JAK2 (Santa Cruz Biotechnology Inc.; rabbit polyclonal antibody), 1:1,000 STAT3 (Millipore; rabbit polyclonal antibody), 1:5,000 p-STAT3 (Abcam; rabbit monoclonal antibody). After membranes were washed with PBS×4, membranes were incubated with 1:8,000 HRP conjugated secondary antibody (Amersham, Biosciences) for 1 hour at room temperature on a rocker. The membranes were washed with PBS×4 and blots were visualized in enhanced Chemiluminescent (ECL) solution (Pierce) for 5 minutes and exposed to hyperfilms (Kodak) for 15 minutes. Blots were again incubated in a Stripping buffer (Pierce) for 15 minutes at room temperature on a rocker and reprobed with 1:12,000 anti-β-actin antibody (Abeam Inc.) as a loading control. Western blots were made in triplicates. Band density was measured and normalized against a loading control band.

### ELISA

Under pentobarbital anesthesia, whole blood was collected from the left ventricle of the heart and placed in a serum collection tube (BD Vacutainer). Blood clots were formed after 30-60 min at room temperature and the tube was centrifuged for 10 min at 12,000 rpm. ELISA Quantikine kit (R&D Systems) and IDO ELISA kit (TSZ ELISA) was used according to the manufacturer's instructions. Optical density of samples was read at wavelengths of 450 & 570 nm using a microplate reader (Synergy HT, Bio-TEK®). A standard curve, provided by the manufacturer, was generated for each set of samples assayed and the concentration of each sample was obtained according to the standard curve.

### Real-Time RT PCR

Total RNA was isolated from hippocampal samples using TRIzol reagent (Invitrogen). Four μg of total RNA was used to synthesize the first strand cDNA using SuperScript III kit (Invitrogen). Four μl of cDNA was put into 20 μl vessel with 1 μl of 20× TaqMan Gene Expression Assay. Taqman Gene Expression Assays (Applied Biosystems) containing primers and a Taqman probe were used to quantify each gene of interest. The genes examined were IL-6 (Rn01410330 m1, M26744.1) and IDO1 (Rn00576778 m1, AF312699.1) for rats and IL-6 (Mm00446190 m1, X54542.1) and IDO1 (Mm00492586 m1, M69109.1) for mice. The reaction was performed in duplicate with the following conditions: denaturing at 95° C. for 30 s, annealing at 60° C. for 2 min, extension at 68° C. for 2 min for 40 cycles using a 7300 Real-Time PCR System (Applied Biosystems). The RNA expression of GAPDH was measured as control.

### HPLC

The hippocampal tryptophan, kynurenine, and serotonin content were determined by HPLC (Widner, B., Werner, E. R., Schennach, H., Wachter, H., and Fuchs, D. 1997. Simultaneous measurement of serum tryptophan and kynurenine by HPLC. Clin. Chem. 43:2424-2426; Laich, A., Neurauter, G., Widner, B., and Fuchs, D. 2002. More rapid method for simultaneous measurement of tryptophan and kynurenine by HPLC. Clin. Chem. 48:579-581). All samples were taken between 2-5 PM. Kynurenine was measured by a UV detector (Shimadzu, SPD-10Avp; 360 nm wavelength). Tryptophan and serotonin were measured by a fluorescence detector (Shimadzu, RF-10Axl; 286 nm excitation and 366 nm emission wavelength). Between different days, external calibration was made by using freshly prepared control samples of same concentration (100 nmol/L tryptophan, 100 μmol/L kynurenine, and 100 nmol/L 5-HT).

### Statistical Analyses

Repeated measure two-way ANOVA was used and followed by the Tukey test to detect interactions between test time points and groups. Correlations between nociceptive and depressive behavior were determined using the Pearson Correlation coefficient analysis. HPLC, western blot and ELISA data were analyzed using one-way ANOVA followed by the Tukey or Dunn's test. The statistical analyses were performed using SigmaStat (version 11) with the significance level of p<0.05.

### Example 1

### Persistent Nociception Induces Depressive Behavior

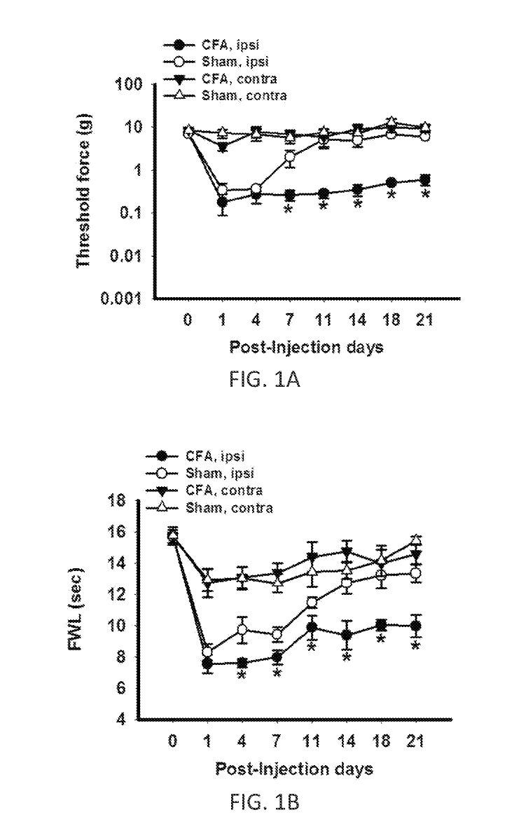

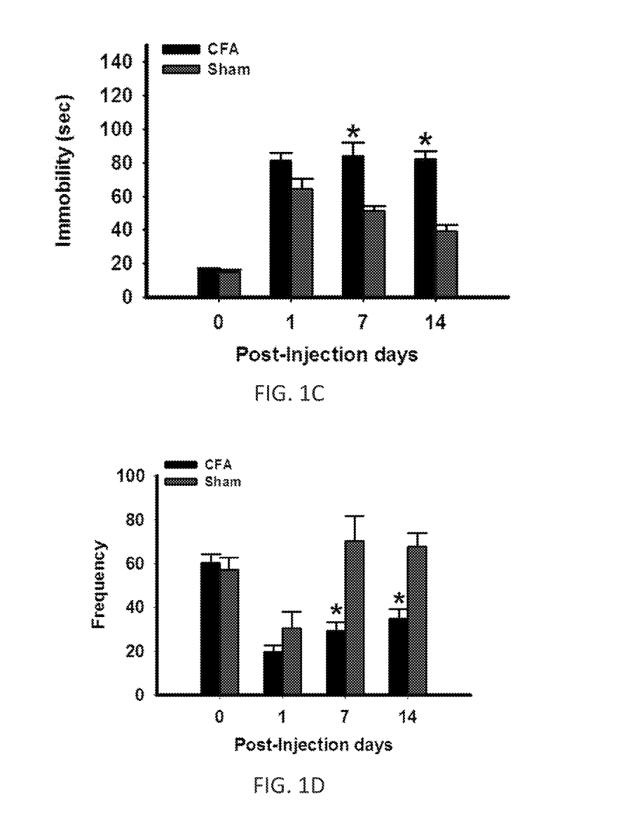

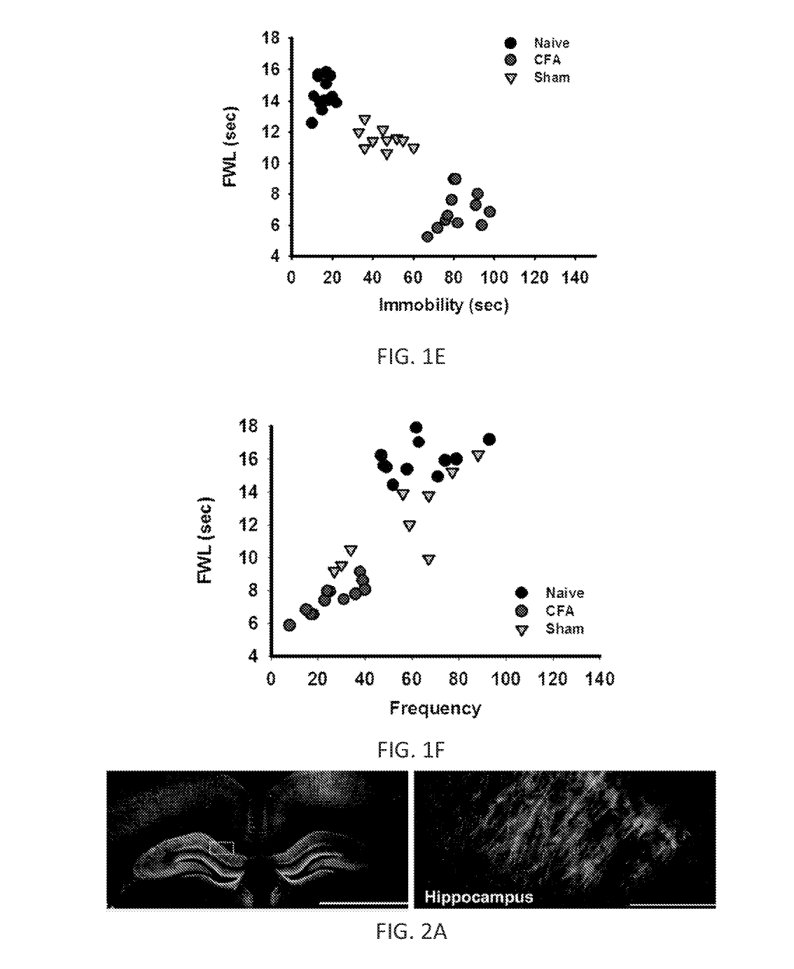

The correlation of nociceptive and depressive behaviors in Wistar rats was examined. Inflammatory arthritis in Wistar rats induced by the injection of complete Freund's adjuvant (CFA) into the right tibio-tarsal joint produced mechanical allodynia [FIG. 1A; ANOVA, F(3,104)=3.11, P<0.05] and thermal hyperalgesia [FIG. 1B; F(3,121)=8.99, P<0.05), which lasted for at least 21 days as compared with sham control rats injected with incomplete Freund's adjuvant. This condition of persistent nociception induced depressive behavior in these same rats when examined on day 7 and 14, but not on day 1, using the forced swimming test (FST) [FIG. 1C; F(3,37)=18.91; P<0.01; see also Porsolt, R. D., Le, P.M., and Jalfre, M. 1977. Depression: a new animal model sensitive to antidepressant treatments. Nature 266:730-732.] and open field test (OFT) [FIG. 1D; F(3,31)=6.08, P<0.05; see also Hall CS 1934. Emotional behavior in rat. J Comp Physiol Psychol 18:385-403 and Pare, W. P. 1994. Open field, learned helplessness, conditioned defensive burying, and forced-swim tests in WKY rats. Physiol Behav. 55:433-439]. A shorter hindpaw withdrawal latency in arthritic rats correlated with a longer immobility time in FST (FIG. 1E) and a lower frequency in OFT (FIG. 1F), demonstrating a comorbid relationship between pain and depression in these rats. Of note, the increased immobility time in FST and reduced frequency in OFT were observed in both arthritic and sham control rats on day 1 but only in arthritic rats on day 7 and day 14. Testing of depressive behavior was not extended beyond day 14 in order to avoid habituation to the testing environment because there were no differences after day 14 in nociceptive behavior. The intensity of exploratory behavior (e.g., rearing and crossing in OFT) was similar between arthritic and sham control rats, although arthritic rats had a lower frequency of exploratory behaviors. Moreover, there were no differences in a rotarod test between rats with or without hindpaw arthritis on day 7 (data not shown), suggesting that the observed depressive behavior was unlikely due to changes in motor function.

### Example 2

### Hippocampal IDO1 Expression is Up-Regulated in Rats with Co-Existent Nociceptive and Depressive Behavior

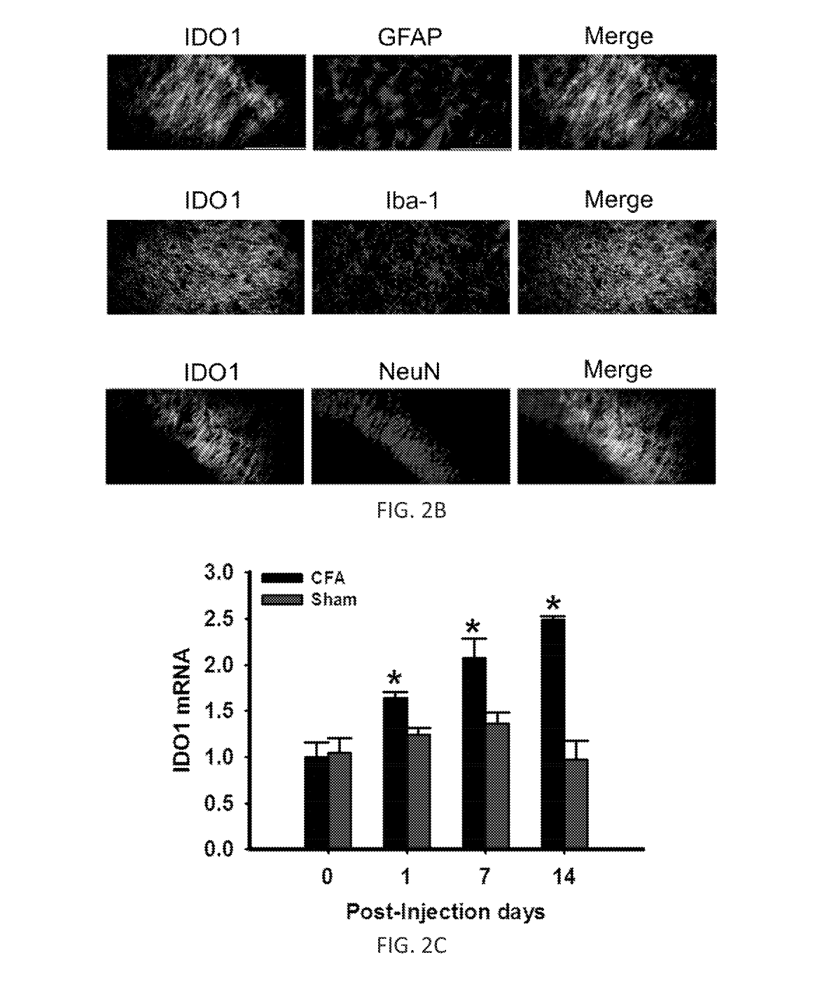

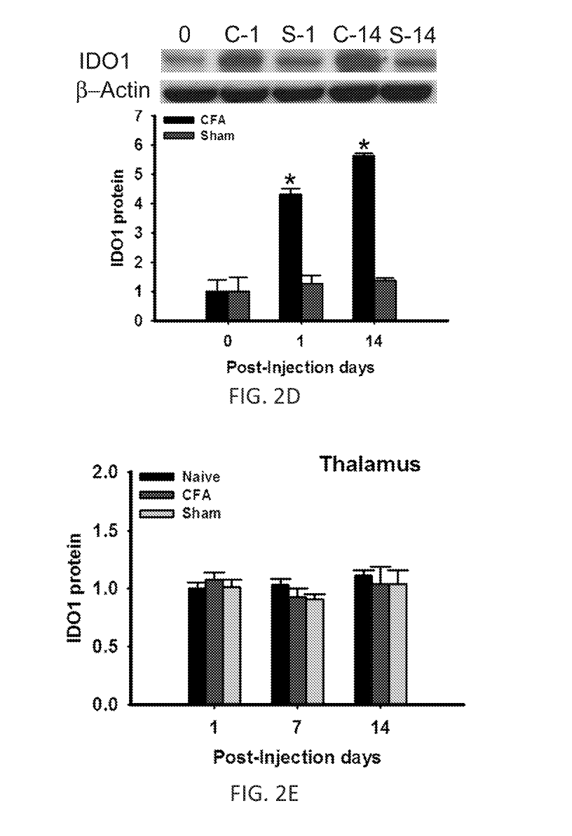

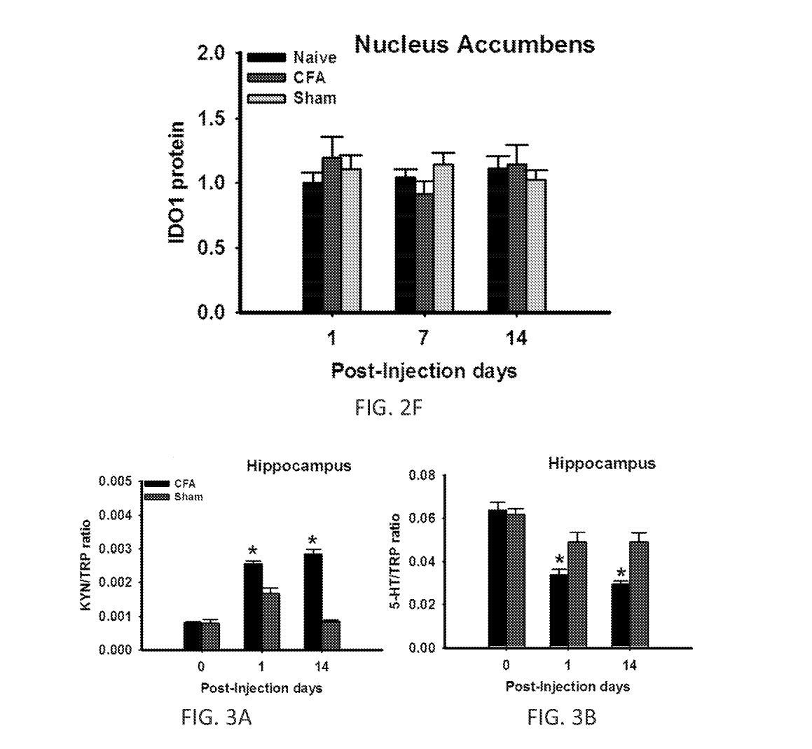

This example examined whether the brain IDO1 expression (hippocampus, thalamus, and nucleus accumbens) would differ in rats with or without co-existent nociceptive and depressive behavior. IDO1 immunoreactivity in the hippocampus (FIG. 2A) was co-localized with GFAP (astrocyte marker), Iba-1 (microglial marker), and NeuN (neuronal marker) (FIG. 2B), consistent with both in vivo and in vitro expression of IDO in immune cells and neurons (Guillemin, G. J., Smythe, G., Takikawa, O., and Brew, B. J. 2005. Expression of indoleamine 2,3-dioxygenase and production of quinolinic acid by human microglia, astrocytes, and neurons. Glia 49:15-23; Roy, E. J., Takikawa, O., Kranz, D. M., Brown, A. R., and Thomas, D. L. 2005. Neuronal localization of indoleamine 2,3-dioxygenase in mice. Neurosci. Lett. 387:95-99). While the basal IDO1 mRNA level (real-time PCR) in the bilateral hippocampus was similar between arthritic and sham rats (naive, day 0; contralateral side only), the IDO1 mRNA level was progressively increased on day 1, 7 and 14 in arthritic but not sham rats (FIG. 2C; P<0.05). The IDO1 protein level (Western blot) was also elevated in the hippocampus (FIG. 2D; contralateral side only, P<0.05), but not in the thalamus or nucleus accumbens (FIGS. 2E and 2F), of arthritic rats. Moreover, there was a temporal relationship between IDO up-regulation and nociceptive (FIGS. 1A and 1B) and depressive behavior (FIGS. 1C and 1E) in these same rats.

### Example 3

### Increased IDO1 Enzyme Activity Alters Ratios of Hippocampal Tryptophan Metabolites

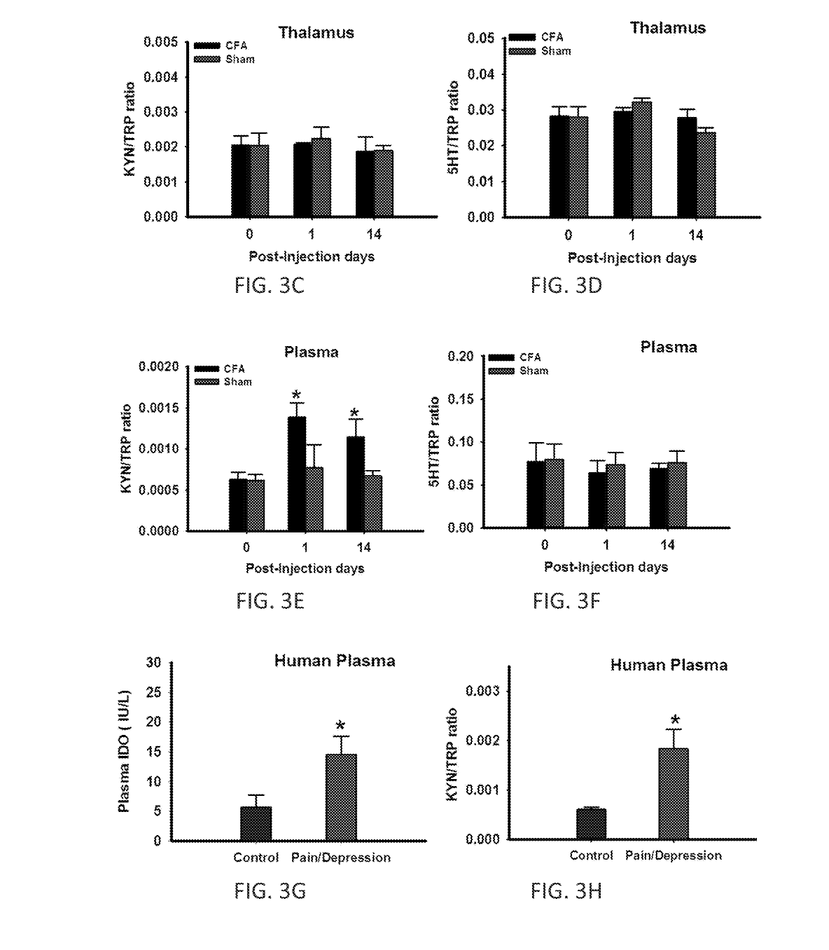

To examine the role of IDO1 enzyme activity in tryptophan metabolism in both arthritic and sham rats, the content of tryptophan, serotonin, and kynurenine in the hippocampus was measured using HPLC and then the ratio of serotonin or kynurenine over tryptophan was determined. There were no baseline differences in the kynurenine/tryptophan or serotonin/tryptophan ratio between arthritic and sham control rats (FIGS. 3A and 3B). However, the kynurenine/tryptophan ratio was significantly increased (FIG. 3A; P<0.05), while the serotonin/tryptophan ratio was decreased (FIG. 3B; P<0.05), in arthritic rats as compared with sham control rats when measured on both day 1 and day 14. In contrast, IDO1 enzyme activity (both kynurenine/tryptophan and serotonin/tryptophan ratios) was not changed in the thalamus of arthritic rats (FIGS. 3C and 3D). In addition, the plasma kynurenine/tryptophan ratio was also significantly increased (FIG. 3E; P<0.05) in arthritic rats as compared to sham control rats, although the plasma serotonin/tryptophan ratio remained unchanged (FIG. 3F), when both examined on day 14. Consistent with the IDO1 up-regulation in arthritic rats, altered ratios of tryptophan metabolites in the hippocampus indicate an increased IDO1 enzyme activity in those rats with co-existent nociceptive and depressive behavior.

Patients with both chronic back pain and depression also showed a significantly elevated plasma IDO level (FIG. 3G) as well as increased IDO1 enzyme activity (FIG. 3H; increased kynurenine over tryptophan ratio) as compared with healthy control subjects without pain and depression (P<0.05). In contrast, the serotonin over tryptophan ratio was not different between healthy control subjects and patients with both pain and depression (FIG. 3I). Although the data was obtained in a cross-section observational setting, these findings suggest that a relationship could also exist in human subjects between IDO1 activity and combined pain and depression.

### Example 4

### Presence of Anhedonic Behavior Exacerbates Nociceptive Behavior

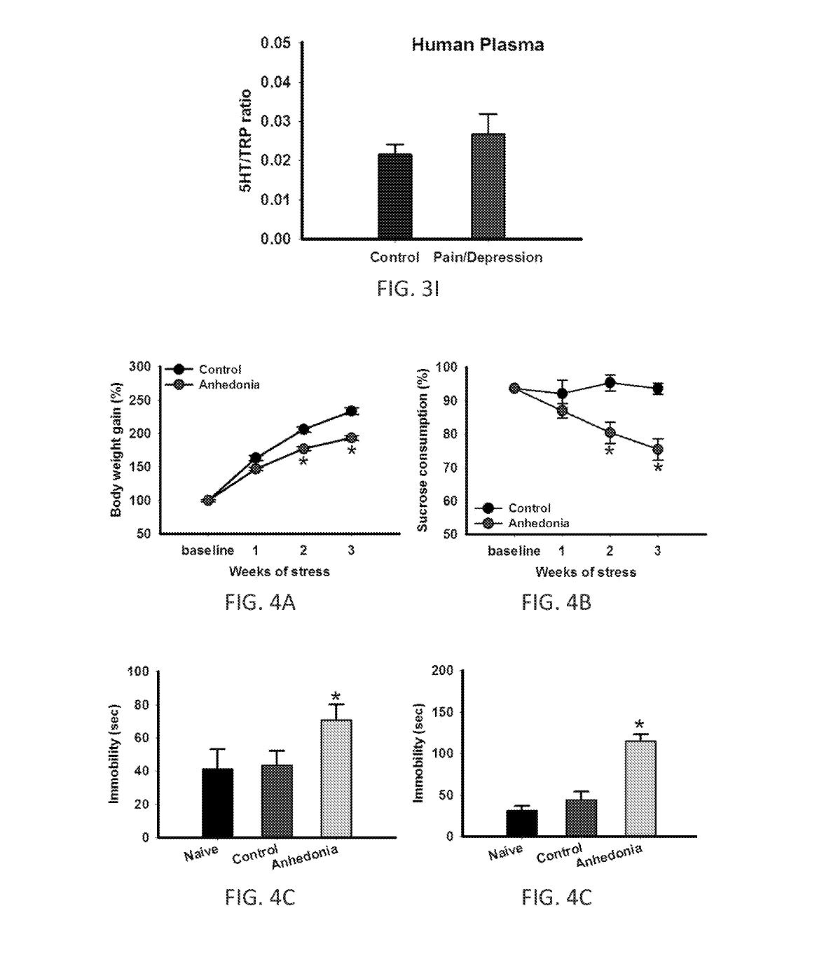

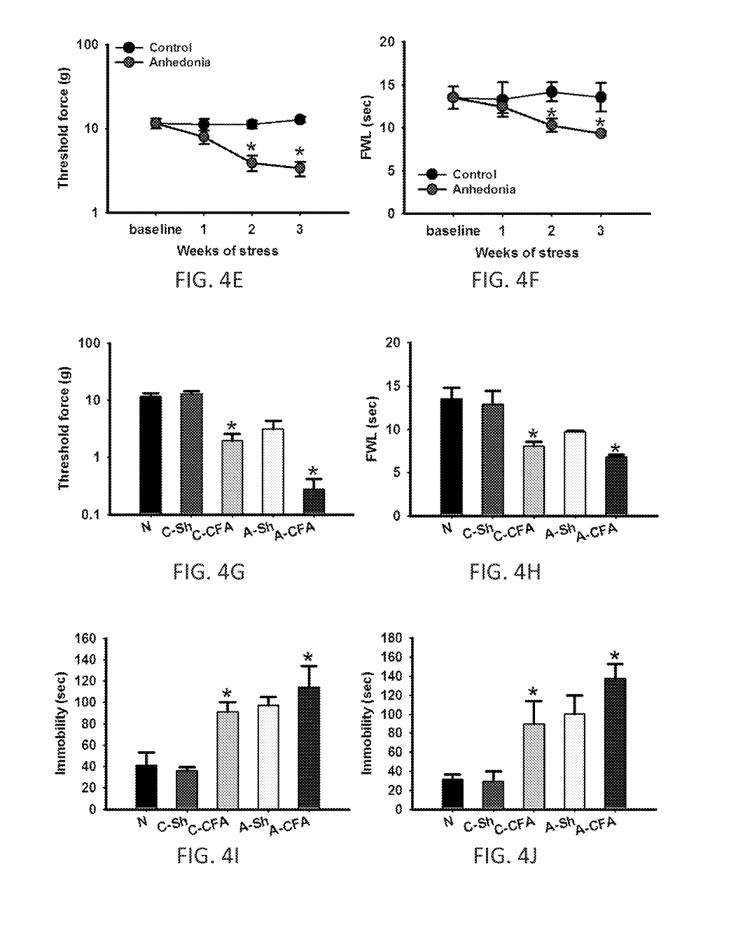

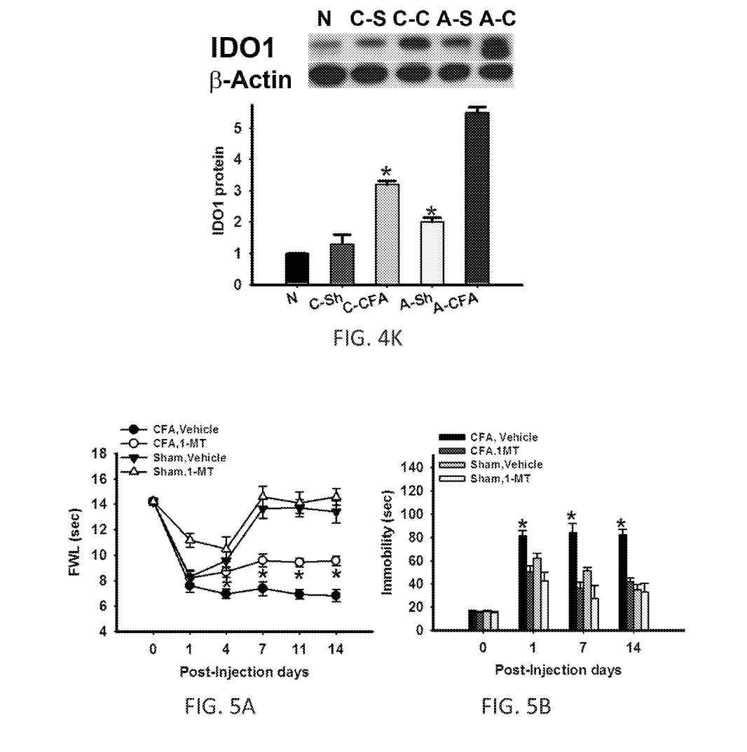

In order to examine the generality of hippocampal IDO1 expression in relation to the interaction between nociception and depression, an established rat model of anhedonia induced by chronic social stress was used (see Methods). After three weeks of chronic social stress, rats demonstrated a significant decrease in body weight gain and sucrose preference than control rats without social stress [FIG. 4A; F(1,23)=553.02, P<0.05 and 4B; F(1,23)=311.83, P<0.05], indicating the presence of anhedonic behavior. These rats also exhibited other depressive behaviors manifesting as a longer immobility time in both FST [FIG. 4C; F (2,35)=31.86, P<0.05] and tail-suspension test (TST) [FIG. 4D; F(2,35)=369.08, P<0.05]. Moreover, these same rats exhibited a progressively lower baseline nociceptive threshold in response to mechanical [FIG. 4E; F(1,23)=312.85, P<0.05] or thermal stimulation [FIG. 4F: F(1,23)=100.276, P<0.05] during three weeks of chronic social stress, indicating that the presence of anhedonic behavior also influenced baseline nociceptive response.

To examine whether pre-existing anhedonic behavior would exacerbate nociceptive behavior following hindpaw arthritis, anhedonic and control rats, following 3 weeks of social stress and sham control respectively, were exposed to either CFA hindpaw arthritis or sham control and examined behavioral changes one week later. Both mechanical allodynia [FIG. 4G; F(4,35)=164.98, P<0.05] and thermal hyperalgesia [FIG. 4H; F(4,35)=63.72, P<0.05] were exacerbated in anhedonic rats as compared to control rats, associated with a significantly longer immobility time in FST [FIG. 4I; F(4,35)=63.19, P<0.05] and TST [FIG. 4J; F(4,33)=73.74, P<0.05] in these same rats.

Moreover, the IDO1 expression (Western blot) in the bilateral hippocampus was significantly increased in anhedonic rats as compared to control rats with or without hindpaw arthritis (FIG. 4K; contralateral side only, P<0.05). These results indicate that nociceptive behavior was exacerbated in rats with pre-existing anhedonic behavior, which was also associated with the up-regulation of IDO1 expression in the hippocampus.

### Example 5

### Inhibition of IDO1 Activity Concurrently Attenuates Nociceptive and Depressive Behavior

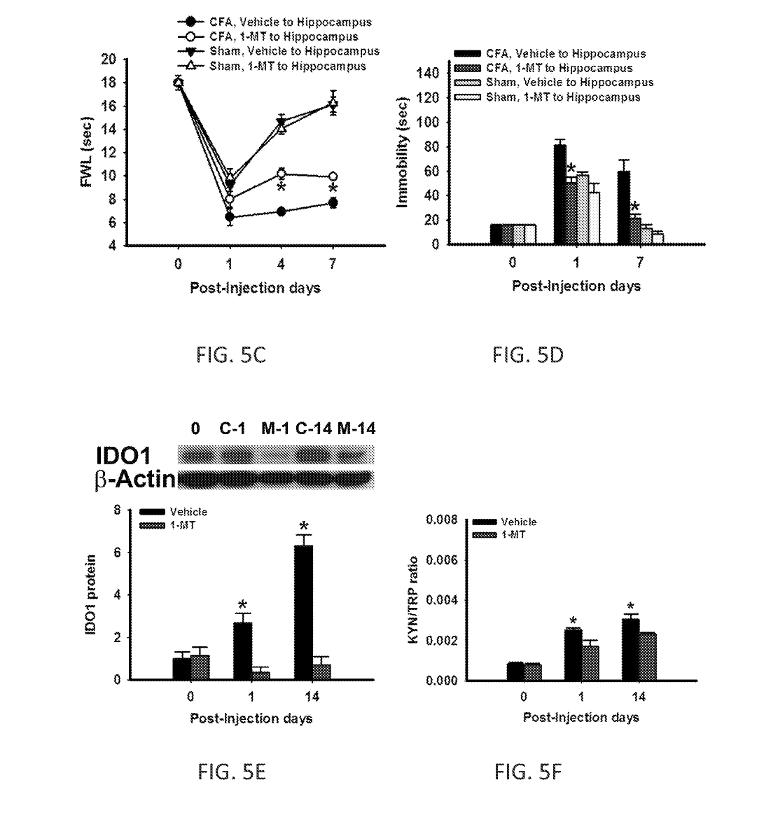

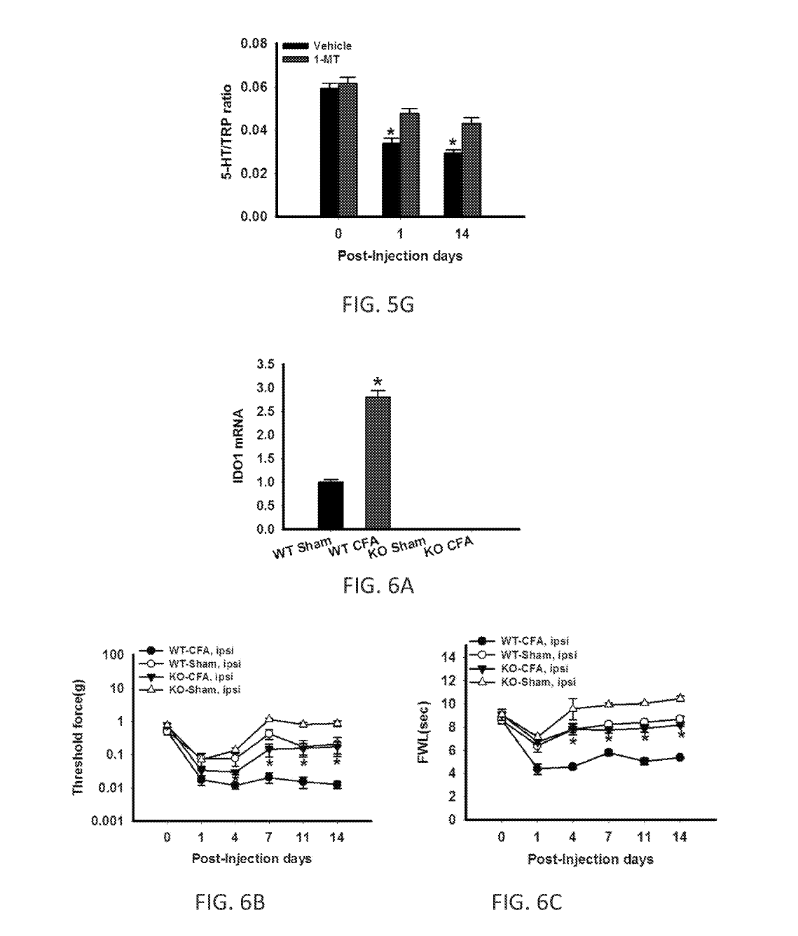

To examine whether inhibition of IDO1 activity would influence nociceptive and depressive behaviors in arthritic rats, the IDO1 inhibitor L-1-methyl-tryptophan (1-MT: 10 mg/day) or vehicle was given intraperitoneally twice daily for 14 consecutive days. Treatment with 1-MT, but not vehicle, significantly attenuated both nociceptive [FIG. 5A; F(3,119)=11.33; P<0.05] and depressive [FIG. 5B: F(3,63)=5.54); P<0.05] behaviors in arthritic rats. Systemic 1-MT treatment alone did not alter behaviors in sham controls rats (FIGS. 5A and 4B), nor did it change the appearance of arthritic hindpaw (e.g., redness or swelling).

To examine the brain site of 1-MT action, 1-MT (5 μg in 0.5 μl volume, once daily×7 d) was micro-injected into the hippocampus contralateral to the arthritic hindpaw. Intra-hippocampal 1-MT treatment also attenuated both nociceptive [FIG. 5C; F(3,71)=5.54, P<0.05] and depressive [FIG. 5D; F(3,31)=14.70, P<0.05] behaviors in arthritic rats without changing behaviors in sham control rats, indicating that the hippocampus is a critical brain locus of IDO1 activity. The procedure of brain cannula implantation itself, used for intra-hippocampal microinjection, did not alter the baseline behavioral response when examined 5 days after the surgery (data not shown).

Intraperitoneal 1-MT treatment also 1) down-regulated IDO1 expression (FIG. 5E, P<0.05), 2) lowered the kynurenine/tryptophan ratio (FIG. 5F, P<0.05), and 3) elevated the serotonin/tryptophan ratio (FIG. 5G, P<0.05) in the hippocampus of arthritic rats. Together with the behavioral data, these results indicate that concurrent attenuation of nociceptive and depressive behavior by the 1-MT treatment was mediated by the regulation of hippocampal IDO1 activity, thereby normalizing the content of tryptophan metabolites in the hippocampus.

### Example 6

### IDO Gene Knockout Attenuates Both Nociceptive and Depressive Behavior

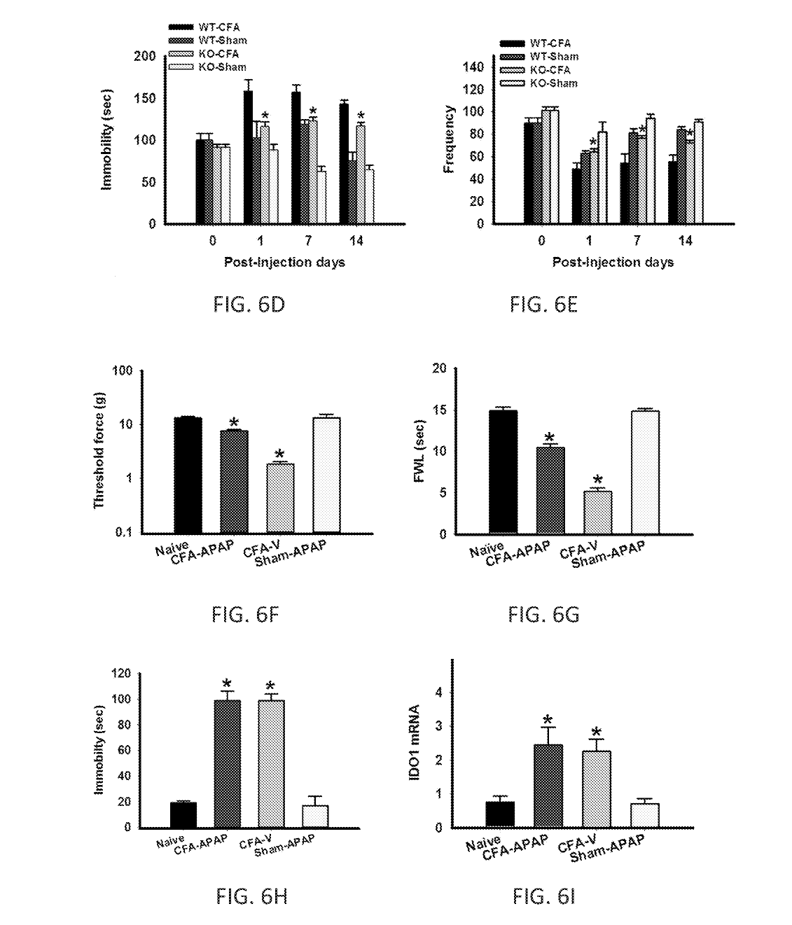

To further confirm the role of IDO1 in the behavioral manifestation of pain and depression, IDO1 knockout and matched wild-type mice were used under the same experimental condition as that for Wistar rats. Both basal and arthritis-induced IDO1 expression in the hippocampus, as observed in age-matched wild-type mice, was absent in IDO1 knockout mice (FIG. 6A). There were no baseline differences in behavioral tests for nociception (FIGS. 6B and 6C; day 0) and depression (FIGS. 6D and 6E; day 0) between IDO1 knockout and wild-type mice (each P>0.05). In IDO1 knockout mice, however, both mechanical allodynia [FIG. 6B; F(3,120)=9.86, P<0.01] and thermal hyperalgesia [FIG. 6C; F(3,122)=5.73. P<0.05] were significantly attenuated as compared with wild-type mice after the CFA injection into the right tibio-tarsal joint. In these same knockout mice, the immobility time (FST) was not increased, nor was there a decrease in the frequency in open field test, as compared with wild-type mice [FIG. 6D; F(3,74)=5.40; 6E; F(3,49)=32.175, each P<0.05]. These results indicate that IDO gene knockout concurrently attenuated nociceptive and depressive behavior induced by persistent hindpaw nociception.

To examine whether selective reduction of nociceptive behavior would influence depressive behavior and hippocampal IDO1 expression, acetaminophen (N-acetyl-para-aminophen, 100 mg/kg), an analgesic agent without the anti-inflammatory effect, or vehicle was given once intraperitoneally on day 14 to arthritic or sham rats. When examined at one hour after the treatment, acetaminophen, but not vehicle, significantly reduced mechanical allodynia [FIG. 6F; F(3,23)=128.80, P<0.05] and thermal hyperalgesia [FIG. 6G; F(3,23)=839.97, P<0.05]. The acetaminophen treatment did not acutely reverse depressive behavior (FIG. 6H; FST), nor did it alter the IDO1 mRNA level in the same arthritic rats (FIG. 6I). These results indicate that the interaction between nociception and depression demonstrated in these rats was not a simple co-existence but linked by the hippocampal IDO expression.

### Example 7

### IL-6 and JAK/STAT are Increased in Rats with Nociceptive and Depressive Behavior

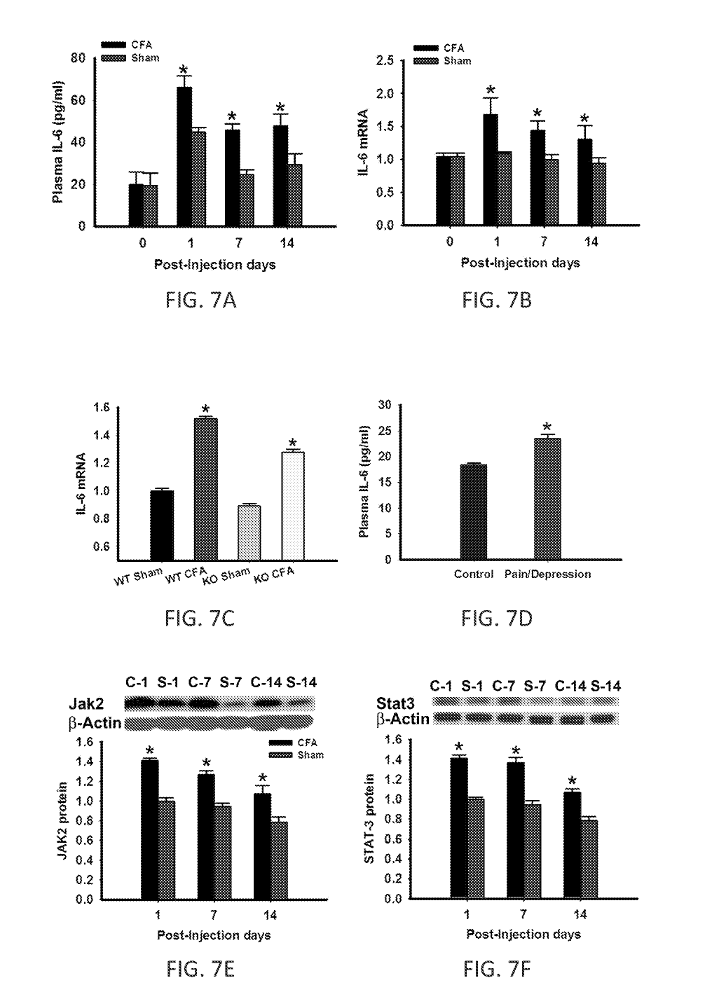

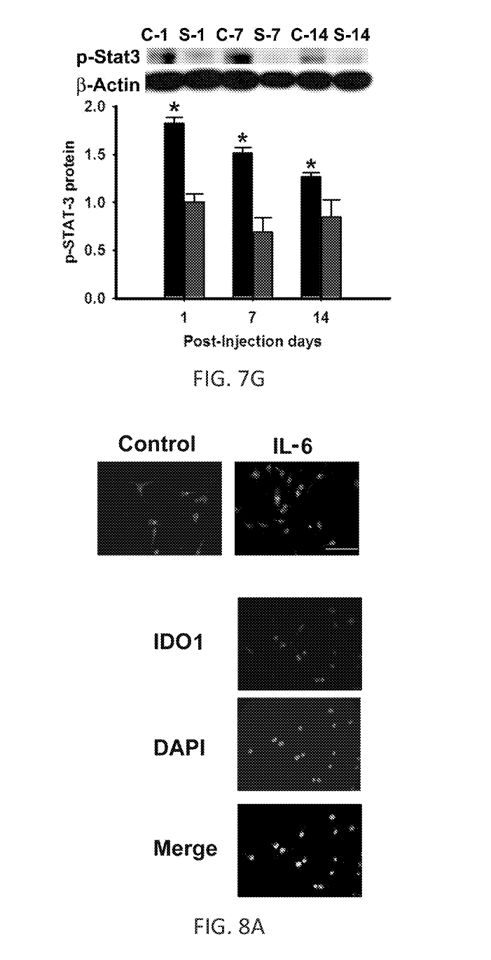

Proinflammatory cytokines including IL-6 have been shown to be involved in the cellular mechanisms of both pain and depression. To examine the hypothesis that proinflammatory cytokines such as IL-6 and one of its downstream signaling pathways [Janus kinase (JAK) and signal transducer and activator of transcription (STAT)] would mediate the hippocampal IDO1 up-regulation, whether the IL-6 level and JAK/STAT expression would be increased in rats with co-existent nociceptive and depressive behavior was examined. Both the plasma IL-6 level and hippocampal IL-6 mRNA expression were significantly increased in rats with nociceptive and depressive behavior as compared with sham rats (FIGS. 7A and 7B: P<0.05). The hippocampal IL-6 mRNA level was also elevated in IDO1 knockout and wild-type mice after the CFA injection into a tibio-tarsal joint (FIG. 7C; P<0.05), indicating that the IL-6 increase was upstream to IDO1 up-regulation. In patients with both chronic pain and depression, the plasma IL-6 content was also elevated as compared with healthy control subjects (FIG. 7D; P<0.05). Of note, the plasma IL-6 content in human subjects was measured in a cross-section observational setting and could be influenced by the subjects' underlying pain condition and other variations such as body weight. Moreover, the expression of IL-6 signaling elements, including JAK2, STAT3, and phosphorylated STAT3 (p-STAT3), was all elevated in the hippocampus of rats with nociceptive and depressive behavior as compared with sham controls (FIG. 7E-G; each P<0.05).

### Example 8

### IL-6 Induces In Vitro IDO1 Up-Regulation

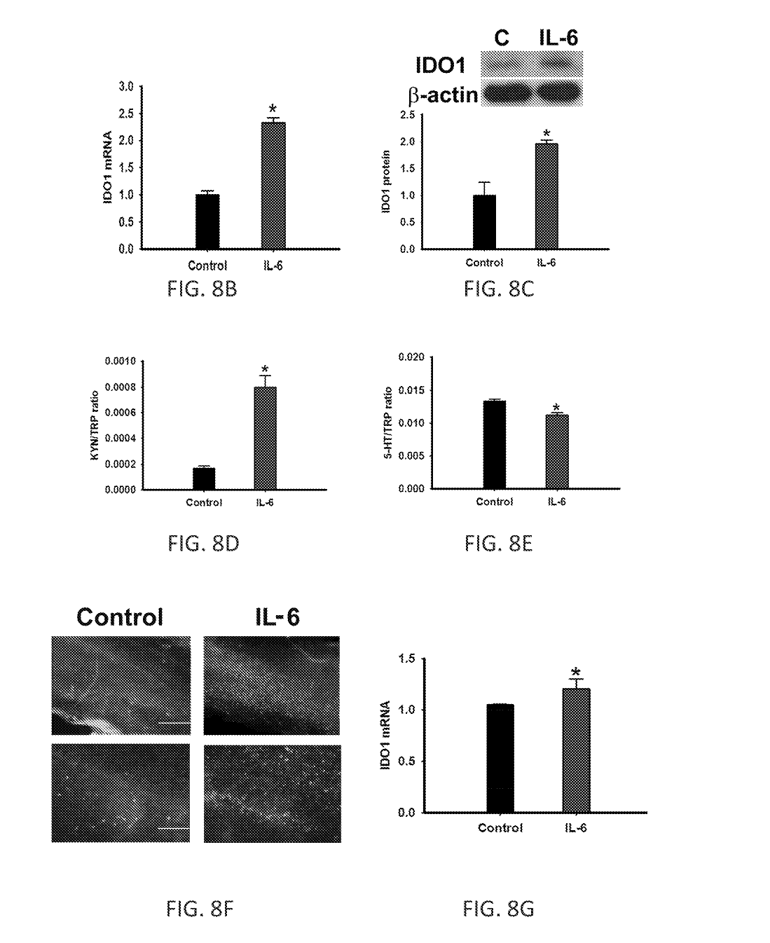

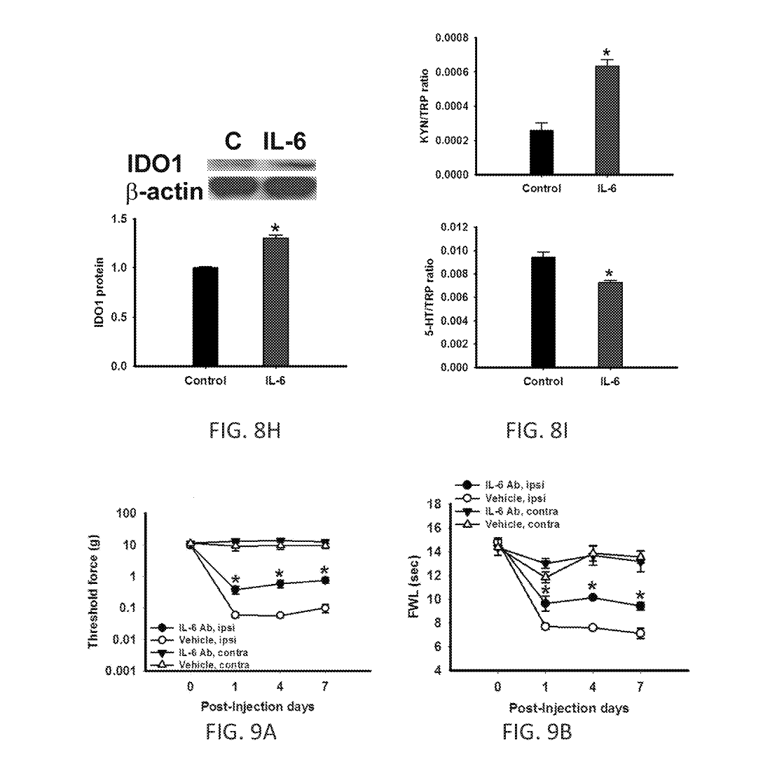

To examine a direct relationship between IL-6 and IDO1 expression at the cellular level, cultured Neuro2a cells were exposed to exogenous IL-6 (0.5 ng/ml) or vehicle (PBS) for 24 hours. IDO1 immunoreactivity was detected in the perinuclear cytoplasm of Neuro2a cells and increased following exposure to IL-6 for 24 hours (FIG. 8A). Exposure of cultured Neuro2a cells to exogenous IL-6, not vehicle, significantly increased the IDO1 mRNA (real-time PCR) and protein (Western blot) expression (FIGS. 8B and 8C; P<0.05), resulting in the increased kynurenine/tryptophan ratio and decreased serotonin/tryptophan ratio (HPLC) in these Neuro2a cells (FIGS. 8D and 8E; P<0.05).

Furthermore, we used a hippocampal organotypic slice culture taken from postnatal rats to examine the in vitro effect of IL-6 on hippocampal IDO1 expression and activity. After being cultured for 1 week, hippocampal slices were treated with IL-6 (100 ng/ml) or vehicle (PBS) for 24 hours. Exposure of exogenous IL-6, but not vehicle, increased IDO1 immunoreactivity (FIG. 8F) and up-regulated the expression of IDO1 mRNA (real-time PCR, FIG. 8G; P<0.05) and protein (Western blot, FIG. 8H; P<0.05) in cultured slices. Under the same experimental condition, the kynurenine/tryptophan ratio was significantly increased, whereas the serotonin/tryptophan ratio was decreased in the culture medium (HPLC, FIG. 8I; P<0.05). Collectively, the results indicate that IL-6 has a direct cellular effect on IDO1 expression in the hippocampus.

### Example 9

### IL-6-Mediated Hippocampal IDO1 Expression Concurrently Regulates Nociceptive and Depressive Behavior

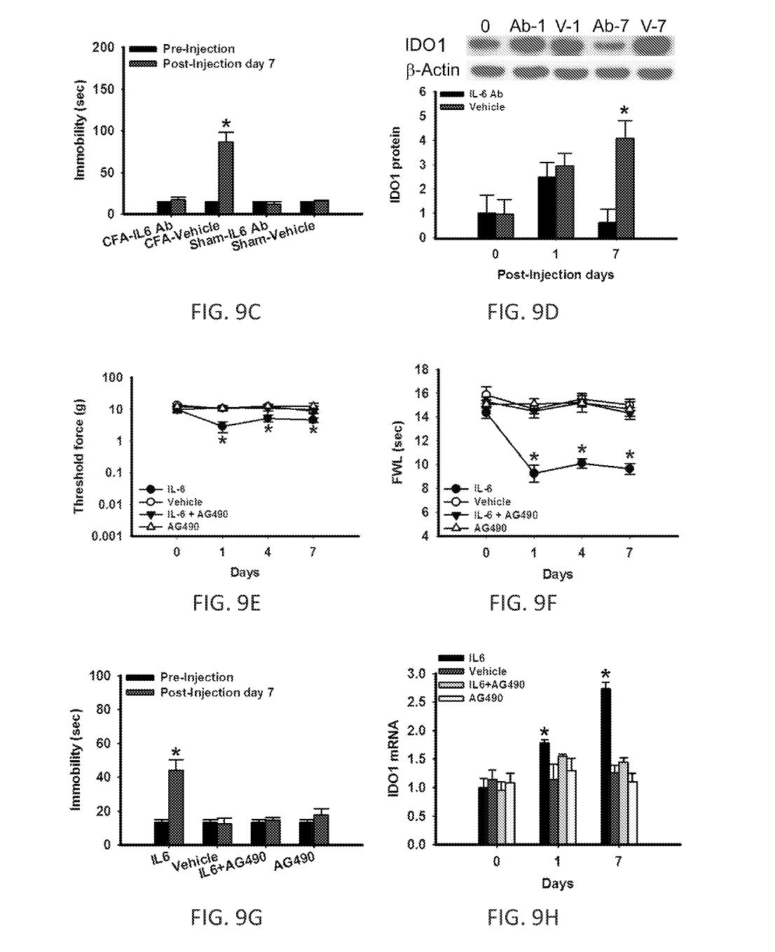

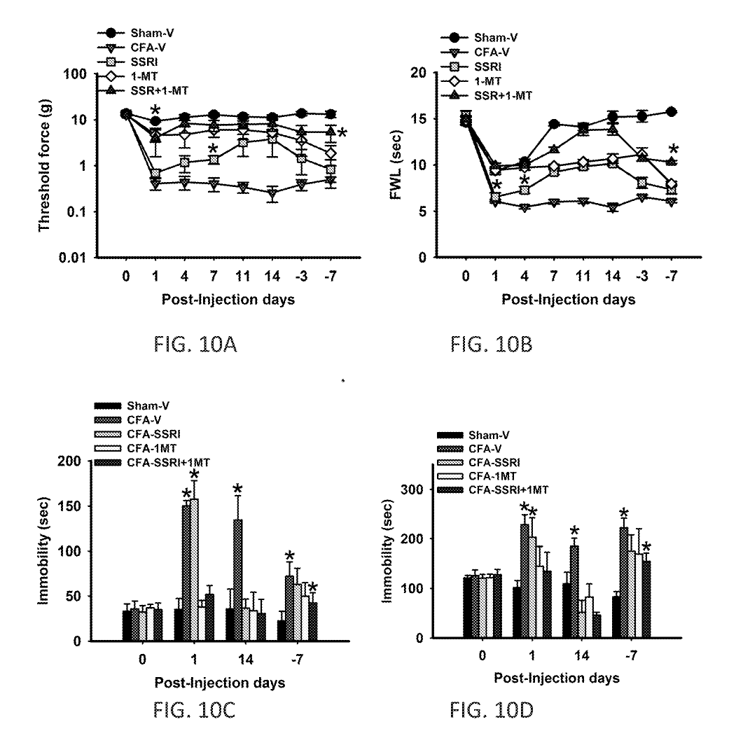

To examine the functional role of IL-6 signaling in the hippocampal IDO1 expression as well as its contribution to both nociceptive and depressive behavior, we micro-injected an IL-6 antiserum into the hippocampus of arthritic or sham control rats. Microinjection of IL-6 antiserum (0.5 μg, once daily×7 d), but not control serum (vehicle), into the hippocampus contralateral to arthritic hindpaw significantly attenuated mechanical allodynia [FIG. 9A; F(1,39)=9.28, P<0.05], thermal hyperalgesia [FIG. 9B; F(1,39)=7.46, P<0.05], and depressive behavior [FIG. 9C; F(3,19)=155.99, P<0.001]. The same IL-6 antiserum treatment also prevented IDO1 up-regulation in the hippocampus (FIG. 9D; P<0.05), consistent with the in vitro results of IL-6-induced IDO1 expression (FIGS. 8B and 8C).

Conversely, microinjection of exogenous IL-6 (recombinant rat IL-6, 0.1 μg, once daily×7 d)(25), but not vehicle, into the left hippocampus of naïve rats (without arthritis) induced right hindpaw mechanical allodynia [FIG. 9E: F(3,79)=2.54, P<0.05] and thermal hyperalgesia [FIG. 9F: F(3,103)=11.24, P<0.01] as well as depressive behavior [FIG. 9G: F(3,19)=65.20, P<0.001] and increased IDO1 mRNA expression in the hippocampus (FIG. 9H; P<0.05). These IL-6 effects were prevented when IL-6 was co-administered with the JAK/STAT inhibitor AG490 (5 μg, once daily×7d) (Dominguez, E., Rivat, C., Pommier, B., Mauborgne, A., and Pohl, M. 2008. JAK/STAT3 pathway is activated in spinal cord microglia after peripheral nerve injury and contributes to neuropathic pain development in rat. J Neurochem. 107:50-60) into the hippocampus (FIG. 9E-H; each P<0.05). Intra-hippocampal microinjection of AG490 alone had no effect on the baseline behavioral response and IDO1 mRNA expression in naive rats (FIG. 9E-H; each P>0.05).

Taken together with the data obtained using the IDO1 inhibitor 1-MT, these findings indicate that the hippocampus is a central site of IL-6-regulated IDO1 expression critically contributory to the comorbid interaction between pain and depression.

### Example 10

### Effect of Drug Combination on Pain and Depression

Rats were divided into five groups: 1) sham-operated rats treated with a vehicle (Sham-V), 2) CFA-induced hindpaw pain treated with vehicle (CFA-V), 3) CFA-induced hindpaw pain treated with fluoxetine (5 mg; CFA-SSRI or SSRI), 4) CFA-induced hindpaw pain treated with the IDO1 inhibitor 1-MT (10 mg, CFA-1MT or 1MT), and 5) CFA-induced hindpaw pain treated with both fluoxetine (5 mg) and 1-MT (10 mg) (CFA-SSRI+1MT or SSR+1MT). Each treatment was given intraperitoneally once per day from day 1 to day 14. Behavioral tests for pain are shown in panel A (mechanical allodynia) and panel B (thermal hyperalgesia). Behavioral tests for depression are shown in panel C (forced swimming test) and panel D (tail-suspension test). Day (−3) and Day (−7) indicate the time point at 3 and 7 days after discontinuation of the corresponding drug treatment.

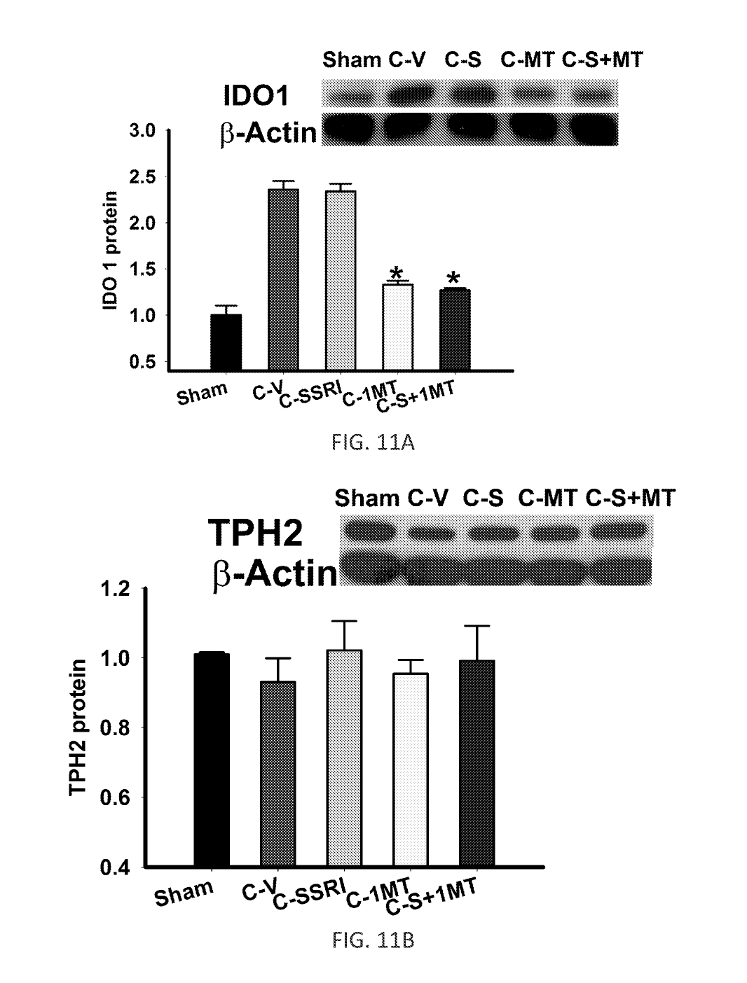

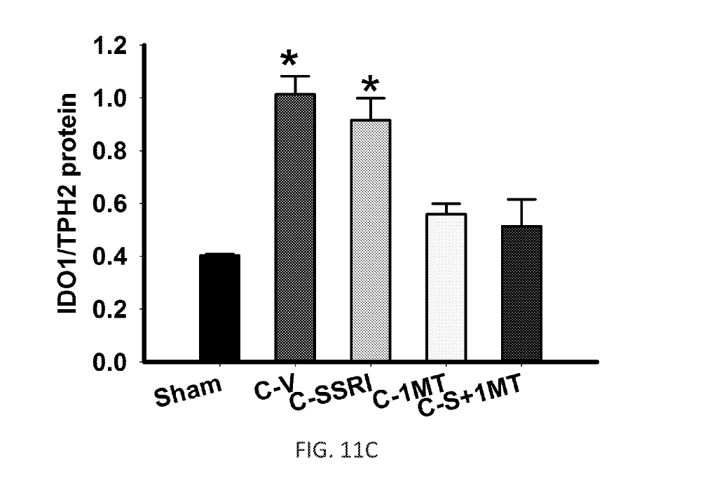

The data indicate that the combination of SSRI with IDO1 inhibitor produces an improved relief in pain and depression with a quicker onset and a prolonged time course even after the discontinuation of the drug treatment. * P<0.05, as compared with the non-combination groups (in A & B) or with the sham group (in C & D). (FIG. 11)

### Example 11

### Altered Ratio of IDO1/TPH2 Expression in the Hippocampus and Normalization by 1-MT Alone and in Combination with SSRI

Rats were divided into five groups: 1) sham-operated rats treated with a vehicle (Sham), 2) CFA-induced hindpaw pain treated with vehicle (C-V). 3) CFA-induced hindpaw pain treated with fluoxetine (5 mg; C-S or C-SSRI), 4) CFA-induced hindpaw pain treated with the IDO inhibitor 1-MT (10 mg, C-1MT or C-MT), and 5) CFA-induced hindpaw pain treated with both fluoxetine (5 mg) and 1-MT (10 mg) (C-S+MT or C-S+1MT). Each treatment was given intraperitoneally once per day from day 1 to day 14. Hippocampal tissue samples were taken after the last behavioral tests at 7 days after discontinuation of the corresponding drug treatment.

The data indicate that IDO1 expression was up-regulated, whereas TPH2 expression unchanged, in the hippocampus of rats with CFA-induced pain, resulting in an increased IDO1/TPH2 ratio. (FIG. 11) Either 1-MT treatment alone or in combination with SSRI prevented the IDO up-regulation and normalized the IDO1/TPH2 ratio. In contrast, SSRI alone did not alter the IDO1/TPH2 ratio. (FIG. 11)

The examples provided herein demonstrate that brain (neuronal) indoleamine 2,3-dioxygenase (IDO), plays a key mechanistic role in comorbid relationship between pain and depression. Persistent hindpaw inflammatory pain in rats induced depressive behavior and IDO1 up-regulation in the bilateral hippocampus but not thalamus or nucleus accumbens. Up-regulation of IDO resulted in the increased kynurenine/tryptophan ratio and decreased serotonin/tryptophan ratio in the bilateral hippocampus. Plasma IDO activity was also elevated in patients with both pain and depression as well as in rats with anhedonia induced by chronic social stress. At the cellular level, interleukin-6 (IL-6) induced IDO1 expression in both Neuro2a cells and an organotypic hippocampal tissue culture, whereas intra-hippocampal administration of IL-6 and its antiserum respectively induced and blocked the hippocampal IDO expression through an intracellular JAK/STAT pathway. Either IDO gene knockout or inhibition of hippocampal IDO1 activity with L-1-methyl-tryptophan attenuated both nociceptive and depressive behavior in the same rodents. These results reveal an endogenous IDO-mediated regulatory mechanism underlying the comorbid interaction between pain and depression and provide a new strategy for the concurrent treatment of both conditions via modulating brain IDO activity.

The data from human subjects provided herein suggests a relationship between IDO expression/enzyme activity and clinical symptoms of pain and depression, but this cross-sectional clinical observation dose not explore the causal relationship between IDO and clinical conditions. On the other hand, the data from animal experiments identify a novel mechanistic link between pain and depression via a critical role of IDO1 in the hippocampus. Regulation of hippocampal IDO1 is likely to be mediated through an IL-6 signal transduction pathway because 1) up-regulation of IL-6 and its downstream JAK and STAT3 preceded the IDO expression in rats with combined nociceptive and depressive behavior; 2) the hippocampal IL-6 mRNA level was elevated in IDO gene knockout mice in response to inflammatory arthritis; 3) inhibition of IDO1 activity by systemic 1-MT treatment did not prevent elevation of the plasma IL-6 level in rats with both nociceptive and depressive behavior (data not shown); and 4) IL-6 directly up-regulated IDO1 expression in both Neuro2a cells and an organotypic hippocampal tissue culture. The examples also indicate that the hippocampus is a critical brain region of IDO regulation because IDO1 was selectively up-regulated in the hippocampus, but not in the thalamus or nucleus accumbens. These findings are consistent with the previous reports that a) certain brain regions including the hippocampus play a critical role in the integration of mood changes and pain and b) hippocampus is related to nociceptive perception and its exacerbation by mood disorders such as anxiety.

Kynurenine and serotonin are two major tryptophan metabolites via enzymatic regulation including IDO, which have been implicated in the mechanisms of pain and depression. The examples indicate that both kynurenine/tryptophan and serotonin/tryptophan ratios in the hippocampus were closely regulated by IDO1 activity. This regulatory mechanism appears to have two important functional implications: on the one hand, increased IDO activity lowers the endogenous serotonin level, which leads to depression and diminishes the descending inhibition of pain modulation; on the other hand, increased IDO activity increases kynurenine derivatives such as quinolinic acid contributing to neurotoxicity and nociception via the interaction with glutamate receptors. Therefore, IDO is situated in a key tryptophan metabolic pathway and alteration of IDO activity results in changes in the content of endogenous kynurenine and serotonin, both of which play a critical role in the mechanisms of pain and depression. This notion is supported by our data showing that concurrent improvement of pain and depression was achieved by inhibiting IDO1 activity or IDO gene knockout, which normalized the increased kynurenine/tryptophan ratio and decreased serotonin/tryptophan ratio resulting from hippocampal IDO up-regulation. It would be of interest in future studies to examine the relationship between IDO expression and other products of tryptophan metabolism and its role in pain and depression.

Studies in the immunology field have consistently shown a relationship between inflammatory mediators and IDO expression in immune cells. Studies using central administration of cytokines have indicated a role for cytokines in various behavioral manifestations. For example, intracerebroventricular (i.c.v.) administration of IL-6 or IL-1β elicited hyperalgesia as well as fever, anorexia, and reduction of social exploratory behavior. Three recent studies including ours have shown that peripheral nerve injury induced depressive behavior in rats, which is associated with an increased IL-1β expression in the frontal cortex. The present data demonstrate a direct link between cytokine signaling and IDO expression in the hippocampus. Given that IDO changes were also demonstrated in a rat model of anhedonia, regulation of brain IDO expression may have a broad implication in the interaction between pain and depression. It will be of considerable interest in future studies to determine whether a similar cytokine and IDO link would be relevant to other pain conditions such as neuropathic pain.

The data from animal experiments identify a novel mechanistic link between pain and depression via a critical role of IDO1 in the hippocampus. Regulation of hippocampal IDO1 is likely to be mediated through an IL-6 signal transduction pathway because 1) up-regulation of IL-6 and its downstream JAK and STAT3 preceded the IDO expression in rats with combined nociceptive and depressive behavior; 2) the hippocampal IL-6 mRNA level was elevated in IDO gene knockout mice in response to inflammatory arthritis: 3) inhibition of IDO1 activity by systemic 1-MT treatment did not prevent elevation of the plasma IL-6 level in rats with both nociceptive and depressive behavior (data not shown); and 4) IL-6 directly up-regulated IDO1 expression in both Neuro2a cells and an organotypic hippocampal tissue culture. The examples also indicate that the hippocampus is a critical brain region of IDO regulation because IDO1 was selectively up-regulated in the hippocampus, but not in the thalamus or nucleus accumbens. These findings are consistent with the previous reports that a) certain brain regions including the hippocampus play a critical role in the integration of mood changes and pain and b) hippocampus is related to nociceptive perception and its exacerbation by mood disorders such as anxiety.

Further, the examples presented herein support a central (hippocampal) effect of IL-6-mediated IDO activity on the behavioral interaction between pain and depression. First, intra-hippocampal microinjection of the IDO1 inhibitor 1-MT attenuated both nociceptive and depressive behavior similar to that after systemic 1-MT administration. Second, neither systemic 1-MT, nor intra-hippocampal administration of IL-6, IL-6 antiserum, or 1-MT, changed signs of hindpaw inflammation (e.g. redness, swelling), suggesting that the effect of 1-MT on nociceptive and depressive behavior is unlikely to be mediated through a peripheral mechanism at the site of hindpaw arthritis. Third, the plasma IDO activity, reflected by an increased kynurenine/tryptophan ratio, was only transiently increased on day 1 but not day 7 and 14 after hindpaw inflammation. Fourth, exogenous IL-6 directly up-regulated IDO1 expression and enhanced IDO activity in Neuro2a cells and an organotypic hippocampal tissue culture. Fifth, intra-hippocampal microinjection of IL-6 in naive rats induced hippocampal IDO up-regulation as well as nociceptive and depressive behavior, which was blocked by AG490 (a JAK/STAT inhibitor). Thus, the examples indicate an important role of IDO activity in the central nervous system in addition to its critical role in the immunoregulation.

Clinical studies have demonstrated that the plasma IL-6 level was increased in patients suffering from painful neuropathy, cancer, inflammation and depression. The examples demonstrate that the plasma IL-6 and IDO level, as well as IDO enzyme activity, was increased in patients with chronic back pain and depression, consistent with the findings from animal studies. This raises the possibility that concurrent treatment of both pain and depression might be possible through regulating brain IDO activity, in contrast to the current approach of symptomatic management using antidepressants and analgesics. Although the neural and cellular mechanism underlying the interaction between pain and depression is likely to be complex and involves other neurotransmitters and neuromodulators, the present findings provide a new strategy of clinical intervention. This new strategy focuses on both prevention and reversal of comorbid interactions between pain and depression by targeting its underlying mechanism involving altered ratios of endogenous tryptophan metabolites resulting from up-regulated IDO expression in certain brain regions.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for the concurrent treatment of pain and depression in a patient in need thereof, comprising administering to the patient an indoleamine 2,3-dioxygenase inhibitor.

2. The method of claim 1, wherein the indoleamine 2,3-dioxygenase inhibitor is selected from 1-methyl-tryptophan (I-MT), β-(3-benzofuranyl)-alanine, β-(3-benzo(b)thienyl)-alanine, or 6-nitro-D-tryptophan.

3. The method of claim 1, further comprising administering a serotonin reuptake inhibitor.

4. The method of claim 3, wherein the serotonin reuptake inhibitor is a selective serotonin reuptake inhibitor (SSRI)

5. The method of claim 3, wherein the serotonin reuptake inhibitor is selected from citalopram, escitalopram, fluoxetine, sertraline, paroxetine, fluvoxamine, venlafaxine, duloxetine, dapoxetine, nefazodone, imipramin, femoxetine, indalpine, zimelidine, and clomipramine.

6. The method of claim 1, further comprising administering tryptophan.

7. The method of claim 1, further comprising a serotonin receptor agonist.

8. The method of claim 1, wherein the indoleamine 2,3-dioxygenase inhibitor is an inhibitor of IDO1.

9. A method of treating pain and depression in an patient, the method comprising administering to the patient an effective amount of an indoleamine 2,3-dioxygenase inhibitor and a serotonin reuptake inhibitor to alleviate the pain and to alleviate at least one symptom of depression.

10. The method of claim 9, wherein the pain is chronic pain selected from lower back pain, a typical chest pain, headache, pelvic pain, myofascial pain, abdominal pain, neck pain and chronic pain caused by a disease or condition.

11. The method of claim 9, wherein a symptom of depression is selected from agitation, restlessness, irritability, depressed mood, diminished interest and pleasure in activities, difficulty concentrating, weight and appetite changes, psychomotor disturbances, sleep disturbances, fatigue and loss of energy, feelings of hopelessness and helplessness, feelings of worthlessness and guilt, concentration difficulties, indecisiveness, thoughts of death or suicide and possibly delusions/hallucinations.

12. The method of claim 9, wherein the indoleamine 2,3-dioxygenase inhibitor is selected from 1-methyl-tryptophan (I-MT), β-(3-benzofuranyl)-alanine, β-(3-benzo(b)thienyl)-alanine, or 6-nitro-D-tryptophan.

13. The method of claim 9, wherein the serotonin reuptake inhibitor is a selective serotonin reuptake inhibitor (SSRI)

14. The method of claim 9, wherein the serotonin reuptake inhibitor is selected from citalopram, escitalopram, fluoxetine, sertraline, paroxetine, fluvoxamine, venlafaxine, duloxetine, dapoxetine, nefazodone, imipramin, femoxetine, indalpine, zimelidine, and clomipramine.

15. The method of claim 9, further comprising administering tryptophan.

16. A method of treating a patient being diagnosed as having both pain and depression, the method comprising identifying a patient diagnosed with both pain and depression; administering to the patient an effective amount of an indoleamine 2,3-dioxygenase inhibitor and a serotonin reuptake inhibitor to alleviate the pain and to alleviate at least one symptom of depression.

17. The method of claim 16, wherein the pain is chronic pain selected from lower back pain, a typical chest pain, headache, pelvic pain, myofascial pain, abdominal pain, neck pain and chronic pain caused by a disease or condition.

18. The method of claim 16, wherein a symptom of depression is selected from agitation, restlessness, irritability, depressed mood, diminished interest and pleasure in activities, difficulty concentrating, weight and appetite changes, psychomotor disturbances, sleep disturbances, fatigue and loss of energy, feelings of hopelessness and helplessness, feelings of worthlessness and guilt, concentration difficulties, indecisiveness, thoughts of death or suicide and possibly delusions/hallucinations.

19. The method of claim 16, wherein the indoleamine 2,3-dioxygenase inhibitor is selected from 1-methyl-tryptophan (I-MT), β-(3-benzofuranyl)-alanine, β-(3-benzo(b)thienyl)-alanine, or 6-nitro-D-tryptophan.

20. The method of claim 16, wherein the serotonin reuptake inhibitor is a selective serotonin reuptake inhibitor (SSRI).

21. The method of claim 16, wherein the serotonin reuptake inhibitor is selected from citalopram, escitalopram, fluoxetine, sertraline, paroxetine, fluvoxamine, venlafaxine, duloxetine, dapoxetine, nefazodone, imipramin, femoxetine, indalpine, zimelidine, and clomipramine.

22. The method of claim 16, further comprising administering tryptophan.
